(19) United States
(12) Patent Application Publication
(10) Pub. No.: US 2024/0148870 A1
(43) Pub. Date: May 9, 2024

(54) ENGINEERED IMMUNE CELLS AND USES THEREOF

(71) Applicant: The Johns Hopkins University, Baltimore, MD (US)

(72) Inventors: Michael S. Hwang, Seattle, WA (US); Jacqueline Douglass, Baltimore, MD (US); Emily Han-Chung Hsiue, Baltimore, MD (US); Kenneth W. Kinzler, Frankford, DE (US); Brian J. Mog, Baltimore, MD (US); Nickolas Papadopoulos, Towson, MD (US); Alexander H. Pearlman, Baltimore, MD (US); Bert Vogelstein, Baltimore, MD (US); Shibin Zhou, Owings Mills, MD (US)

(21) Appl. No.: 18/281,471

(22) PCT Filed: Mar. 11, 2022
(86) PCT No.: PCT/US2022/019980

§ 371 Date: Sep. 11, 2023

Related U.S. Application Data
(60) Provisional application No. 63/160,338, filed on Mar. 12, 2021.

(51) Int. Cl. A61K 39/00; A61P 35/00; C07K 14/705; C07K 14/725; C07K 16/28; C12N 5/0783

(52) U.S. Cl. CPC A61K 39/464412; A61K 39/4611; A61K 39/4631; A61P 35/00; C07K 14/7051; C07K 14/70517; C07K 14/70521; C07K 16/2803; C07K 16/2833; C12N 5/0636; A61K 2239/21; A61K 2239/22; A61K 2239/28; C07K 2317/53; C07K 2317/622; C07K 2319/02; C07K 2319/03; C12N 2510/00

(57) ABSTRACT

Provided herein are immune cells and methods of use, wherein the immune cells include a chimeric antigen receptor (CAR), wherein the CAR comprises an extracellular antigen binding domain that binds specifically to a first epitope; and an inhibitory chimeric antigen receptor (iCAR), wherein the iCAR comprises an extracellular antigen binding domain that binds specifically to a second epitope, wherein the immune cell is activated when the immune cells binds to the first epitope and does not bind to the second epitope; and wherein the immune cell is inactivated when the immune cell binds to the first and second epitopes.

### CROSS-REFERENCE TO RELATED APPLICATIONS

This application claims priority to U.S. Provisional Patent Application No. 63/160,338, filed on Mar. 12, 2021. The disclosure of this prior application is considered part of the disclosure of this application, and is incorporated in its entirety into this application.

### FEDERALLY SPONSORED RESEARCH OR DEVELOPMENT

This invention was made with government support under grants AR048522, CA006973, CA009071, CA230400 and GM073009 awarded by the National Institutes of Health. The government has certain rights in the invention.

### TECHNICAL FIELD

The present disclosure relates to the field of biotechnology, and more specifically, to engineered immune cells.

### SEQUENCE LISTING

This application contains a Sequence Listing that has been submitted electronically as an ASCII text file named 448070389WO1seglisting. The ASCII text file, created on Mar. 11, 2011, is 88.7 kilobytes in size. The material in the ASCII text file is hereby incorporated by reference in its entirety.

### BACKGROUND

Cancer cells differ from non-cancer cells. Despite years of research, however, harnessing the molecular differences between cancer cells and non-cancer cells to selectively target cancer cells to achieve improved treatment regimens and patient outcomes remains an active area of endeavor.

Immune cells (e.g., T cells) engineered with chimeric antigen receptors (CAR-T) have great therapeutic potential for treating diseases such as cancers. CAR-T therapeutics confer powerful target affinity and signaling function on T cell. However, there remains an unmet need to develop immune cells that can detect and specifically target cancer cells while distinguishing them from normal cells.

### SUMMARY

The present disclosure is based on the discovery that engineered immune cells (e.g., T cells) as described herein, can more selectively target a cancer cell, rather than a corresponding non-cancerous cell. The presently provided immune cells can provide for selective treatment of a cancer in a subject and/or a reduction in on-target off-tumor cytotoxicity in non-cancer cells. Without wishing to be bound by any theory, it has been discovered that engineered immune cells can be generated to target gene loss in disease (e.g., a cancer).

Provided herein are immune cells comprising: (a) a chimeric antigen receptor (CAR), wherein the CAR comprises an extracellular antigen binding domain that binds specifically to a first epitope; and (b) an inhibitory chimeric antigen receptor (iCAR), wherein the iCAR comprises an extracellular antigen binding domain that binds specifically to a second epitope, wherein the immune cell is activated when the immune cells binds to the first epitope and does not bind to the second epitope; and wherein the immune cell is inactivated when the immune cell binds to the first and second epitopes. In some embodiments, the immune cell is a T cell.

In some embodiments, the first epitope is expressed from a first allele and the second epitope is expressed from a second allele, wherein the first allele and the second allele are from a same gene. In some embodiments, the first epitope, the second epitope, or both is expressed from a human leukocyte antigen (HLA) gene. In some embodiments, the first epitope is a tumor-associated antigen (TAA). In some embodiments, the second epitope is expressed from a polymorphic allele that is lost in a cancer cell but present in a normal cell. In some embodiments, the first epitope, the second epitope, or both is present in an extracellular domain of a cell surface protein. In some embodiments, the first epitope, the second epitope, or both is present in an intracellular protein that is presented on the surface of a cell by an HLA molecule.

In some embodiments, a transmembrane domain of the CAR comprises a CD8-alpha transmembrane domain. In some embodiments, a hinge region of the CAR comprises a CD28 hinge region. In some embodiments, an intracellular signaling domain of the CAR comprises a CD28 intracellular signaling domain or a CD3-zeta intracellular signaling domain. In some embodiments, a transmembrane domain of the iCAR comprises a CD8-alpha transmembrane domain, a PD-1 transmembrane domain, or a CTLA-4 transmembrane domain. In some embodiments, the hinge region of the iCAR comprises a CD8-alpha hinge region, a PD-1 hinge region, or a CTLA-4 hinge region. In some embodiments, the intracellular signaling domain of the iCAR comprises a PD-1 inhibitory domain or a CTLA-4 inhibitory domain.

In some embodiments, the CAR and the iCAR are conditionally expressed in the immune cell. In some embodiments, the CAR and the iCAR are expressed in the immune cell in a tumor microenvironment. In some embodiments, the immune cell further comprises a synNotch receptor, wherein the synNotch receptor activates the CAR and the iCAR expression when the immune cell is in the tumor microenvironment.

Provided herein are pharmaceutical compositions comprising any one of the immune cells described herein and a pharmaceutically acceptable carrier.

Provided herein are methods of treating a subject having a disease, the method comprising administering to the subject any one of the immune cells described herein, or any one of the pharmaceutical compositions described herein. In some embodiments, the disease is a cancer. In some embodiments, the disease is a precancerous pathology with LOH.

In some embodiments, the subject has previously been administered one or more additional anticancer therapies selected from the group consisting of ionizing radiation, a chemotherapeutic agent, a therapeutic antibody, or a checkpoint inhibitor. In some embodiments, the subject will be administered one or more additional anticancer therapies selected from the group consisting of ionizing radiation, a chemotherapeutic agent, a therapeutic antibody, or a checkpoint inhibitor. In some embodiments, the cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.

In some embodiments, the precancerous pathology is selected from a myelodysplastic syndrome, acquired aplastic anemia, Fanconi anemia, paroxysmal nocturnal hemoglobinuria (PNH), 5q-syndrome, or any condition characterized by pathogenic cells with clonal LOH. In some embodiments, the immune cell targets LOH present in a hematologic malignancy. In some embodiments, the hematologic malignancy is selected from myelodysplastic syndrome (MDS), acute myeloid leukemia (AML), or acute lymphocytic leukemia (ALL). In some embodiments, the hematologic malignancy has relapsed after matched, mis-matched and haploidentical blood or marrow transplantation. In some embodiments, the immune cell targets LOH which are present in cancer cells and cause relapse after targeted CAR T cell therapy or bispecific antibody therapy directed against a tumor associated antigen. In some embodiments, the tumor associated antigen is CD19 or NY-ESO-1.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows an exemplary schematic describing a cellular engineering strategy to target loss of heterozygosity (LOH) events in cancer. Arrows labeled “Allele A” and “Allele B” depict the production of a polymorphic protein as a result of transcription and translation of a polymorphic gene subject to LOH in cancer. The NASCAR (Neoplasm-targeting Allele-Sensing CAR) platform comprises pairwise CAR and iCAR receptors in T cells. Concurrent engagement of both receptors will result in iCAR-mediated quenching of proximal CAR signaling and divert T-cell activation away from normal cells expressing both alleles (left). However, cancer cells that have undergone LOH will trigger the CAR but not the iCAR, resulting in NASCAR T-cell activation (right).

FIG. 2 shows 65 linearized CAR plasmids that were in vitro-transcribed, capped, and tailed, wherein the resultant mRNA was assessed for integrity and expected transcript size by RNA TapeStation gel analysis.

FIG. 3 shows the flow cytometric evaluation of primary human T cells following CAR mRNA electroporation. Five unique CAR mRNAs were electroporated into T cells, while Mock indicates T cells electroporated with no mRNA. The inset image shows brightfield and fluorescent images of T cells following CAR mRNA electroporation. GFP fluorescence was used to assess transfection efficiency.

FIGS. 4A-4C are a set of graphs showing identification of HLA-A3 allele-specific scFvs with phage display technology. FIG. 4A shows results when T cells expressing a CAR grafted with either a GAP.A3 (α-A3) or BB7.2 (α-A2) scFv were co-incubated with target cell lines expressing A2 and A3 (T2A3) or A2 only (T2), and T-cell activation was assessed by ELISA for secreted IFN-γ. FIG. 4B shows results where enriched candidate A3 phage clones were evaluated for binding to cell lines expressing A2 and A3 (T2A3 cells) or A2 only (T2 cells) by flow cytometry. NC indicates no phage control. FIG. 4C shows results where T cells expressing a CAR grafted with either a BB7.2 (α-A2) or Clone 13 (α-A3) scFv were co-incubated with COS-7 cells transfected with the indicated HLA-A or HLA-B allele at an E:T ratio of 1:1. T-cell activation was assessed by ELISA for secreted IFN-γ.

FIGS. 5A-5C show results of generation of HLA-A allele-targeting scFvs and isogenic cell line models. FIG. 5A is a graph wherein A2-scFv binding to various immobilized HLA alleles was assessed by ELISA. FIG. 5B is a graph wherein A3-scFv binding to various immobilized HLA alleles was assessed by ELISA. FIG. 5C shows flow cytometric evaluation with α-A2 (BB7.2-PE) and α-A3 (GAP.A3-APC) antibodies of HLA KO isogenic cancer cell lines following CRISPR-mediated HLA-A locus disruption.

FIG. 6 is a bar graph where pHLA complexes were evaluated for antigen integrity by performing an ELISA using the W6/32 antibody, which recognizes only folded HLA.

FIGS. 7A-7F are exemplary schematics showing a summary of the CAR and iCAR designs employed during NASCAR optimization. FIG. 7A shows a CD28-hinged 2^{nd} generation CAR. FIG. 7B shows a CD8α-hinged 2^{nd} generation CAR. FIG. 7C shows a PD-1 cytoplasmic domain with CD8α hinge and transmembrane domain iCAR. FIG. 7D shows a PD-1 cytoplasmic domain with endogenous PD-1 hinge and transmembrane domain iCAR. FIG. 7E shows a CTLA-4 cytoplasmic domain with CD8α hinge and transmembrane domain iCAR. FIG. 7F shows a CTLA-4 cytoplasmic domain with endogenous CTLA-4 hinge and transmembrane domain iCAR.

FIG. 8 is a bar graph showing results where T cells configured with the indicated CAR and 5 variant iCAR combinations were co-incubated with COS-7 cells transfected with the indicated HLA-A allele(s) at an E:T ratio of 1:1. T-cell activation was assessed by ELISA for secreted IFN-γ.

FIG. 9 is a bar graph where T cells configured with the indicated hinge variant CAR and iCAR combinations were co-incubated with CFPAC-1 HLA KO isogenic cell lines with the indicated HLA-A allele status at an E:T ratio of 1:1. T-cell activation was assessed by ELISA for secreted IFN-γ.

FIG. 10 is a graph showing correlation between electroporated mRNA and CAR expression. CAR expression, as assessed by GFP levels, was evaluated by flow cytometry wherein the indicated amounts of CAR mRNA were electroporated into primary human T cells.

FIG. 11 is a graph showing NASCAR stoichiometric optimization for allele-specific inhibition. T cells configured with the indicated amounts and ratios of CAR and iCAR were co-incubated with CFPAC-1 HLA KO isogenic cell lines with the indicated HLA-A allele status at an E:T ratio of 1:1. T-cell activation was assessed by ELISA for secreted IFN-γ.

FIGS. 12A-12C are a set of bar graphs showing NASCAR specificity in vitro. Three isogenic cancer cell line models of LOH were employed to determine NASCAR specificity. Cancer cells with the indicated HLA-A allele status were co-incubated with CAR or NASCAR T cells configured with the indicated allele-targeting CAR and iCAR receptor combinations at an E:T ratio of 2:1. T-cell activation was assessed by ELISA for human IFN-γ, hIFN-7 (FIG. 12A) and human IL-2, hIL-2 (FIG. 12B) release. Cytotoxicity mediated by CAR or NASCAR T cells was measured by CellTiter-Glo (CFPAC-1, NCI-H441) or Steady-Glo (RPMI-6666) (FIG. 12C).

FIGS. 13A-13B are a set of bar graphs showing assessment of NASCAR autoreactivity and re-challenge in “autologous” T cells. FIG. 13A shows results where A2-CAR T cells or NASCAR T cells targeting A3 loss from two different donors with the indicated HLA-A alleles were independently cultured, and T-cell autoreactivity was assessed by ELISA for secreted IFN-γ. FIG. 13B shows results where A2-CAR T cells or NASCAR T cells targeting A3 loss from two different donors with the indicated HLA-A alleles were co-incubated with CFPAC-1 HLA KO isogenic cell lines with the indicated HLA-A allele status at an E:T ratio of 1:1. T-cell activation, upon re-challenge to CFPAC-1 target cells, was assessed by ELISA for secreted IFN-γ.

FIGS. 14A-14D show generation and allele-specificity of CRISPR-engineered NASCAR T cells. FIG. 14A shows flow cytometric evaluation with Protein L and α-CD3 antibody of CRISPR-engineered A3-CAR T cells or NASCAR T cells targeting A2 loss following knock-in of the indicated expression cassette at the B2M locus, with or without simultaneous TRAC KO. FIG. 14B shows flow cytometric evaluation with A2 or A3 pHLA tetramers of CRISPR-engineered A3-CAR T cells or NASCAR T cells targeting A2 loss following knock-in of the indicated expression cassette at the B2M locus with simultaneous TRAC KO. FIGS. 14C-14D are a set of bar graphs showing results where CRISPR-engineered A3-CAR T cells or NASCAR T cells targeting A2 loss were co-incubated with CFPAC-1 HLA KO isogenic cell lines with the indicated HLA-A allele status at an editing efficiency-corrected E:T ratio of 1:1, and T-cell activation was assessed by ELISA for secreted IFN-7 (FIG. 14C) and IL-2 (FIG. 14D).

FIGS. 15A-15B show evaluation of NASCAR antitumor activity in vivo. FIG. 15A is an exemplary schematic showing a single-flank, subcutaneous (SQ) xenograft model wherein NSG mice were employed, and CRISPR-engineered A3-CAR T cells or NASCAR T cells targeting A2 loss were introduced via tail vein intravenous (IV) injection 10 days following tumor inoculation. Tumors were measured bi-weekly for 75 days following tumor inoculation. FIG. 15B is a set of graphs showing tumor growth curves that were serially monitored by external caliper measurements. Inset graph shows magnified window of the first 45 days of treatment. N=6 mice per group.

FIG. 16 is a set of graphs showing a single-flank, subcutaneous xenograft model wherein NSG mice were employed, and CRISPR-engineered A3-CAR T cells and NASCAR T cells targeting A2 loss were introduced via tail vein IV injection 10 days following tumor inoculation. Body weights of the mice were serially monitored throughout the duration of the 75-day experiment. N=6 mice per group.

FIGS. 17A-17F are exemplary schematics showing next-generation LOH targeting. FIG. 17A shows NASCAR targeting as applied to genetically unlinked molecules. FIG. 17B shows NASCAR targeting as applied to intracellular polymorphic peptides that are presented on the cell surface in the context of HLA molecules. FIGS. 17C-17E show a model of tumor-specific inhibitory markers revealed by LOH that are amenable to NASCAR-based targeting. FIG. 17F shows TAA-specific synNotch receptor-driven conditional expression of a NASCAR expression cassette. TF indicates transcription factor.

FIG. 18 is an exemplary schematic describing the proposed strategy to target LOH events in cancer. Input LOH events are integrated by constitutively-expressed, tandem LOH-sensing Notch receptors to regulate an IL-2 transcriptional output program. The IL-2 expression cassette can be replaced with other desired transgenes (e.g. CAR, therapeutic antibodies, cytotoxic agents, etc.).

FIG. 19 is a bar graph showing synNotch-NASCAR activity assessed by ELISA for IL-2. CFPAC-1 cancer cells with the indicated HLA-A allele status were co-incubated with synNotch-NASCAR T cells configured with the indicated allele-targeting synNotch activator and/or repressor mRNA(s) and IL-2 DNA response element combinations.

FIGS. 20A-20C are exemplary schematics of paired CAR constructs and iCAR variants. FIG. 20A shows an exemplary schematic where the CAR construct is composed of an scfv, CD8alpha hinge domain, CD28 transmembrane and intracellular domain, and a CD3zeta intracellular domain. The iCAR is composed of an scfv, CD8alpha hinge and transmembrane domain, and a PD-1 intracellular domain. FIG. 20B shows an exemplary schematic where the CAR construct is composed of an scfv, CD28 hinge/transmembrane/intracellular domains, and a CD3zeta intracellular domain. The iCAR is composed of an scfv, CD8alpha hinge and transmembrane domain, and a PD-1 intracellular domain. FIG. 20C show an exemplary schematic where the CAR construct is composed of an scfv, human CD8alpha hinge and transmembrane domains, CD28 intracellular domain, and a CD3zeta intracellular domain. The two iCARs displayed both have an scfv attached to a mouse CD8alpha hinge domain (to prevent heterodimerization). One iCAR variant includes a mouse CD8alpha transmembrane domain connected to a SUP-1 domain. The other iCAR variant includes a LAIR1 transmembrane and intracellular domain and a separate chimeric molecule in which a PTPN6 SH2 domain is linked to a CD148 phosphatase domain.

FIG. 21 shows exemplary schematics of CAR and iCAR homology directed repair templates, wherein CAR and iCAR constructs are designed to be introduced into T cells as dsDNA templates via homology directed repair at the B2M locus after a CRISPR Cas9 induced double stranded DNA break. Major components such as homology arms, 2A sequences, CAR, iCAR, and polyA terminators are labeled.

FIGS. 22A-22B show flow characterization of CAR and iCAR constructs, wherein CAR and iCAR constructs were introduced into primary human T cells via electroporation of a Cas9 RNP targeted to the B2M locus and dsDNA homology directed repair templates. T cells were assayed via flow cytometry 6 days after initial electroporation. FIG. 22A shows protein L labeling of single live cells (labels A3-CAR). FIG. 22B shows A2 and A3 tetramer staining of single, live cells.

FIGS. 23A-23C are a set of bar graphs showing co-culture assessment of CAR and iCAR combinations. IL-2 (FIG. 23A) and IFNg (FIG. 23B) in cell culture supernatants were quantified via ELISA. FIG. 23C shows IL-2 and IFNg concentrations secreted by only the CD8hinge/transmembrane constructs.

FIG. 24A is an exemplary schematic of homology directed repair templates (HDRTs) designed to skew CAR:iCAR expression ratios.

FIGS. 24B-24C show results where modified T cells were stained with an A3 tetramer to detect CAR expression and an A2 tetramer to detect iCAR expression via flow cytometry. All constructs were expressed from an endogenous B2M promoter in FIG. 24B, whereas comparisons between an endogenous B2M promoter and an exogenous EF1a promoter are shown in FIG. 24C.

### DETAILED DESCRIPTION

The present disclosure is based on the discovery that engineered immune cells (e.g., T cells) as described herein, can more selectively target a cancer cell, rather than a corresponding non-cancerous cell. The presently provided immune cells can provide for selective treatment of a cancer or premalignant pathologies (e.g. myeloid disorders such as myelodysplastic syndromes) in a subject and/or a reduction in on-target off-tumor cytotoxicity in non-cancer cells, where it has been discovered that engineered immune cells can be generated to target loss of a cell surface antigen in disease (e.g., a cancer). In some embodiments, immune cells (e.g., T cells) engineered with chimeric antigen receptors (CAR-T) can have great therapeutic potential for treating diseases such as cancers. CAR therapeutics may confer powerful target affinity and signaling function on T cells, while there remains an unmet need to develop immune cells that can detect and specifically target cancer cells while distinguishing them from normal cells.

In some embodiments, provided herein are immune cells that include a CAR, wherein the CAR comprises an extracellular antigen binding domain that binds specifically to a first epitope, and an inhibitory chimeric antigen receptor (iCAR), wherein the iCAR comprises an extracellular antigen binding domain that binds specifically to a second epitope (e.g., an epitope that is not present on a surface of a cancer cell). In some embodiments, the immune cell is activated when the immune cells binds to the first epitope and does not bind to the second epitope. In some embodiments, the immune cell is inactivated when the immune cell binds to the first and second epitopes.

Various non-limiting aspects of these immune cells are described herein, and can be used in any combination without limitation. Additional aspects of various components of immune cells are known in the art.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies, polyclonal antibodies, and fragments thereof. In some embodiments, an antibody agent may include one or more sequence elements are humanized, primatized, chimeric, etc. as is known in the art. In many embodiments, the term “antibody agent” is used to refer to one or more of the art-known or developed constructs or formats for utilizing antibody structural and functional features in alternative presentation. For example, in some embodiments, an antibody agent utilized in accordance with materials and methods provided herein is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof, single chain Fvs (scFvs); polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent is or comprises at least a portion of a chimeric antigen receptor (CAR). In some embodiments, an antibody agent is or comprises a T cell receptor (TCR).

As used herein, the term “antigen” refers to an agent that binds to an antibody agent. In some embodiments, an antigen binds to an antibody agent and may or may not induce a particular physiological response in an organism. In general, an antigen may be or include any chemical entity such as, for example, a small molecule, a nucleic acid, a polypeptide, a carbohydrate, a lipid, a polymer (including biologic polymers [e.g., nucleic acid and/or amino acid polymers] and polymers other than biologic polymers [e.g., other than a nucleic acid or amino acid polymer]) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some certain embodiments, an antigen is present in a cellular context (e.g., an antigen is expressed on the surface of a cell or expressed in a cell). In some embodiments, an antigen is a recombinant antigen.

As used herein, “antigen-binding domain” refers to an antibody agent or portion thereof that specifically binds to a target moiety or entity. Typically, the interaction between an antigen binding domain and its target is non-covalent. In some embodiments, a target moiety or entity can be of any chemical class including, for example, a carbohydrate, a lipid, a nucleic acid, a metal, a polypeptide, or a small molecule. In some embodiments, an antigen binding domain may be or comprise a polypeptide (or complex thereof). In some embodiments, an antigen binding domain is part of a fusion polypeptide. In some embodiments, an antigen binding domain is part of a chimeric antigen receptor (CAR). In some embodiments, an antigen binding domain is part of a T cell receptor (TCR).

As used herein, the term “binding” typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

As used herein, the terms “cancer”, “malignancy”, “neoplasm”, “tumor”, and “carcinoma” refer to cells that exhibit relatively abnormal, uncontrolled, and/or autonomous growth, so that they exhibit an aberrant growth phenotype characterized by a significant loss of control of cell proliferation. In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, a relevant cancer may be characterized by a hematologic tumor. In general, examples of different types of cancers known in the art include, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, precancerous pathology such as myelodysplastic syndromes, acquired aplastic anemia, Fanconi anemia, paroxysmal nocturnal hemoglobinuria (PNH) and 5q-syndrome and the like.

As used herein, the term “chemotherapeutic agent” can refer to one or more pro-apoptotic, cytostatic and/or cytotoxic agents, for example specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some embodiments, a chemotherapeutic agent may be utilized in the context of an antibody-drug conjugate.

As used herein, the terms “chimeric antigen receptor” and “CAR” are used interchangeably, and refer to engineered immune receptors capable of triggering or inhibiting the activation of an immune cell. In some embodiments, a CAR comprises an extracellular antigen binding domain (e.g., a ligand/antigen-binding domain), a transmembrane domain and one or more intracellular signaling domains. Exemplary CARs, exemplary domains within CARs, and derivatives thereof (e.g., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety. In some embodiments, a CAR can include an inhibitory chimeric antigen receptor (iCAR). As used herein, the term “inhibitory chimeric antigen receptor” refers to engineered immune receptors capable of inhibiting the activation of an immune cell. In some embodiments, an iCAR comprises an extracellular antigen binding domain (e.g., a ligand/antigen-binding domain), a transmembrane domain and one or more intracellular inhibitory signaling domains. In some embodiments, the intracellular signaling domain limits T cell responsiveness. In some embodiments, the inhibiting effect of the iCAR is temporary. In some embodiments, the inhibiting effect of the iCAR is reversible. In some embodiments, the inhibiting effect of the iCAR is permanent. In some embodiments, the inhibiting effect of the iCAR is irreversible.

As used herein, the term “engineered” refers to the aspect of having been manipulated by the hand of man. For example, a polypeptide is considered to be “engineered” when the polypeptide sequence manipulated by the hand of man. For example, in some embodiments of the present invention, an engineered polypeptide comprises a sequence that includes one or more amino acid mutations, deletions and/or insertions that have been introduced by the hand of man into a reference polypeptide sequence. In some embodiments, an engineered polypeptide includes a polypeptide that has been fused (i.e., covalently linked) to one or more additional polypeptides by the hand of man, to form a fusion polypeptide that would not naturally occur in vivo. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, derivatives and/or progeny of an engineered polypeptide or cell are typically still referred to as “engineered” even though the actual manipulation was performed on a prior entity.

As used herein, the term “epitope” refers to a portion of an antigen that is specifically bound by an antigen-binding domain through a set of physical interactions between: (i) all monomers (e.g. individual amino acid residues, sugar side chains, and post-translationally modified amino acid residues) on the portion of the antigen-binding domain that specifically binds the antigen and (ii) all monomers (e.g. individual amino acid residues, sugar side chains, post-translationally modified amino acid residues) on the portion of the antigen that is specifically bound by the antigen-binding domain. Epitopes can, e.g., consist of surface-accessible amino acid residues, sugar side chains, phosphorylated amino acid residues, methylated amino acid residues, and/or acetylated amino acid residues and may have specific three-dimensional structural characteristics, as well as specific charge characteristics. Conformational and non-conformational epitopes are distinguished in that the binding to the former, but not the latter, may be lost in the presence of denaturing solvents. In some embodiments, an epitope is defined by a linear amino acid sequence of at least about 3 to 6 amino acids, or about 10 to 15 amino acids. In some embodiments, an epitope refers to a portion of a full-length protein or a portion thereof that is defined by a three-dimensional structure (e.g., protein folding). In some embodiments, an epitope is defined by a discontinuous amino acid sequence that is brought together via protein folding. In some embodiments, an epitope is defined by a discontinuous amino acid sequence that is brought together by quaternary structure (e.g., a cleft formed by the interaction of two different polypeptide chains). The amino acid sequences between the residues that define the epitope may not be critical to three-dimensional structure of the epitope. A conformational epitope may be determined and screened using assays that compare binding of antigen-binding protein construct to a denatured version of the antigen, such that a linear epitope is generated. An epitope may include amino acid residues that are directly involved in the binding, and other amino acid residues, which are not directly involved in the binding. Methods for identifying an epitope to which an antigen-binding domain specifically binds are known in the art, e.g., structure-based analysis (e.g. X-ray crystallography, NMR, and/or electron microscopy) (e.g. on the antigen and/or the antigen-antigen-binding domain complex) and/or mutagenesis-based analysis (e.g. alanine scanning mutagenesis, glycine scanning mutagenesis, and homology scanning mutagenesis) wherein mutants are measured in a binding assay with a binding partner, many of which are known in the art.

As used herein, the term “pharmaceutical composition” refers to a composition in which an active agent is formulated together with one or more pharmaceutically acceptable carriers. In some embodiments, the composition is suitable for administration to a human or animal subject. In some embodiments, the active agent is present in unit dose amount appropriate for administration in a therapeutic regimen that shows a statistically significant probability of achieving a predetermined therapeutic effect when administered to a relevant population.

As used herein, the phrase “present on the surface of a cell” (e.g., a mammalian cell) refers to (i) an antigen that is physically attached to or at least partially embedded in the plasma membrane of a cell (e.g., a transmembrane protein, a peripheral membrane protein, a lipid-anchored protein (e.g., a GPI-anchor), an N-myristolyated protein, or a S-palmitoylated protein) or (ii) an antigen that is bound to its cognate receptor, where the cognate receptor is physically attached to the plasma membrane of a cell (e.g., a ligand bound to its cognate receptor, where the cognate receptor is physically attached to the plasma membrane). Non-limiting methods for determining the presence of antigen on the surface of a cell (e.g., a mammalian cell) include fluorescence-activated cell sorting (FACS), immunohistochemistry, cell-fractionation assays and Western blotting.

As used herein, “single chain variable fragment, scFv” refers to a fragment of antibody defined as a recombinant protein comprising a heavy chain variable domain (VH) and a light chain variable domain (VL) connected by a linker, which brings the two domains together into association such that an antigen-binding site is formed.

As used herein, the term “specific binding” refers to an ability to discriminate between possible binding partners in the environment in which binding is to occur. A binding agent that interacts with one particular target when other potential targets are present is said to “bind specifically” to the target with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

### Engineered Immune Cells

In some embodiments, provided herein are immune cells including (a) a chimeric antigen receptor (CAR), wherein the CAR includes an extracellular antigen binding domain that binds specifically to a first epitope; and (b) an inhibitory chimeric antigen receptor (iCAR), wherein the iCAR includes an extracellular antigen binding domain that binds specifically to a second epitope, wherein the immune cell is activated when the immune cells binds to the first epitope and does not bind to the second epitope; and wherein the immune cell is inactivated when the immune cell binds to the first and second epitopes. In some embodiments, the immune cell is a T cell.

As used herein, “immune cells” refer to cells of the immune system which can be categorized as lymphocytes (e.g., T cells, B cells, NK cells and NKT cells), neutrophils, and monocytes/macrophages. In some embodiments, the immune cell is a T cell. In some embodiments, an immune cell is an engineered immune cell, which means the immune cell has been genetically modified to express a non-naturally occurring protein (e.g., a chimeric antigen receptor) or to include an exogenous nucleic acid.

The immune cells (e.g., T cells) may be modified in one or more than one manner. Immune cells (e.g., T cells) may express at least one non-natural molecule that is a receptor for an antigen that is present on the surface of one or more types of cells. In some embodiments, immune cells, include immune cells (e.g., T cells) that are not found in nature because they are engineered to comprise or express at least one synthetic molecule that is not found in nature. In specific embodiments, the immune cells (e.g., T cells) are engineered to express at least one chimeric antigen receptor (CAR), including a CAR that targets a specific tumor antigen. In some embodiments, the immune cells are engineered to express two or more chimeric antigen receptors (CARs), including a CAR that targets a first epitope (e.g., a tumor associated antigen), and an inhibitory chimeric antigen receptor (iCAR) that targets a second epitope (e.g., an antigen expressed from a polymorphic allele that is lost in a cancer cell but present in a normal cell).

In specific embodiments, the immune cell can be, without limitation, a T cell, e.g., a CD4+ T cell, a CD8+ T cell, a Treg cell, a Th1 T cell, a Th2 T cell, a Th17 T cell, an unspecific T cell, or a population of T cells that comprises a combination of any of the foregoing, a natural killer T (NKT) cell, a natural killer (NK) cell, or a macrophage. Immune cells (e.g., T cells) engineered with chimeric antigen receptors (CAR T cells) have great therapeutic potential for treating cancers. With a CAR, a receptor can be programmed to recognize an antigen, which when bound, activate immune cells to kill the cell expressing that antigen. Therefore, immune cells expressing CAR(s) for an antigen expressed on a tumor cell can target and kill the tumor cell. For example, recent clinical trials of a CD19-targeted CAR-transduced T cell (CD19-CAR T cell) against hematologic malignancies showed a strong effect of CAR T technology. (Kochenderfer, J. N. et al. (2010) Blood 116: 4099-4102; Porter, D. L., et al. (2011) N. Engl. J. Med. 365: 725-733; Grupp, S. A. et al. (2013) N. Engl. J. Med. 368: 1509-1518; Kochenderfer, J. N. et al. (2015) J. Clin. Oncol. 33: 540-549; Brown, C. E. et al. (2016) N. Engl. J. Med. 375: 2561-2569). The clinical success of CAR T is attributed, at least in part, to the fusion structure of the CAR, which is made by artificially combining a high-affinity antigen-binding domain with multiple signaling domains (Maus, M. V. et al. (2014) Blood 123: 2625-2635; van der Stegen, S. J. et al. (2015) Nat. Rev. Drug Discov. 14: 499-509).

In some embodiments, engineered immune cells provided herein are derived from autologous T cells. In some embodiments, engineered immune cells provided herein are derived from immune cells obtained from a subject that is not the patient. In some embodiments, engineered T cells for use in a therapeutic method are syngeneic (the donor and the recipients are different but are identical twins). In some embodiments, engineered T cells for use in a therapeutic method are allogenic (from the same species but different donor) as the recipient subject. In some embodiments, engineered T cells provided herein are derived from autologous stem cells (for autologous stem cell therapy or ASCT). In some embodiments, engineered immune cells provided herein are derived from non-autologous T cells. In some embodiments, engineered immune cells provided herein are derived from immune cells obtained from a healthy donor. In some embodiments, engineered immune cells provided herein are derived from immune cells obtained from a patient afflicted with a cancer or a tumor.

### Chimeric Antigen Receptors (CARs)

As is known in the art, CARs typically comprise at least an extracellular antigen-binding domain, a transmembrane domain, a hinge region, and an intracellular signaling domain. In some embodiments, the extracellular antigen-binding domain comprises a single chain variable fragment (scFv) that is capable of recognizing a tumor-associated antigen. In some embodiments, a CAR comprises an extracellular antigen-binding domain, a hinge domain or a spacer, a transmembrane domain, and an intracellular signaling domain. In some embodiments, a CAR further comprises a co-stimulatory domain. In some embodiments, a CAR further comprises two co-stimulatory domains. In some embodiments, a CAR further comprises two or more co-stimulatory domains. In some embodiments, the transmembrane domain includes a CD8-alpha transmembrane domain. In some embodiments, the hinge region includes a CD28 hinge region. In some embodiments, the intracellular signaling domain includes a CD28 intracellular signaling domain or a CD3-zeta intracellular signaling domain. See, e.g., Abate-Daga et al., Molecular Therapy Oncolytics (2016) 3, 16014, the disclosure of which is incorporated herein by reference in its entirety.

Inhibitory Chimeric Antigen Receptors (iCARS)

As is known in the art, inhibitory chimeric antigen receptors (iCARs) comprise at least an extracellular antigen-binding domain, a transmembrane domain, a hinge region, and an intracellular inhibitory signaling domain. In some embodiments, the extracellular antigen-binding domain includes an scFV that is capable of recognizing an antigen expressed from a polymorphic allele that is lost in a cancer cell but present in a normal cell. In some embodiments, the transmembrane domain of the iCAR includes a CD8-alpha transmembrane domain, a PD-1 transmembrane domain, or a CTLA-4 transmembrane domain. In some embodiments, the hinge region of the iCAR includes a CD8-alpha hinge region, a PD-1 hinge region, or a CTLA-4 hinge region. In some embodiments, the intracellular signaling domain of the iCAR comprises a PD-1 inhibitory domain, a CTLA-4 inhibitory domain, or any other domain that can mediate inhibitory activity.

In some embodiments of any of the CARs and/or iCARs described herein, the dissociation equilibrium constant (K_{D}) of the binding affinity for an extracellular antigen-binding domain to an antigen and/or an epitope can be between about 1 pM to about 10 μM (e.g. between about 1×10⁻¹² M to about 1×10⁻⁶ M, 1×10⁻¹² M to about 1×10⁻⁷ M, 1×10⁻¹² M to about 1×10⁻¹ M, 1×10⁻¹² M to about 1×10⁻⁹ M, 1×10⁻¹² M to about 1×10⁻¹⁰ M, 1×10⁻¹² M to about 1×10⁻¹¹ M, 1×10⁻¹¹ M to about 1×10⁻⁶ M, 1×10⁻¹⁰ M to about 1×10⁻⁶ M, 1×10⁻⁹ M to about 1×10⁻⁶ M, 1×10⁻⁸M to about 1×10⁻⁶ M, 1×10⁻⁷ M to about 1×10⁻⁶ M, or any range in between). In some embodiments, the dissociation equilibrium constant (K_{D}) of the binding affinity for an extracellular antigen-binding domain to an antigen and/or an epitope can be less than about 1×10⁻⁵ M, less than about 1×10⁻⁶ M, less than about 1×10⁻⁷ M, less than about 1×10⁻⁸ M, less than about 1×10⁻⁹ M, less than about 1×10⁻¹⁰ M, less than about 1×10⁻¹¹ M, less than about 1×10⁻¹² M, or lower.

In some embodiments, the transmembrane domain for use in CARs and/or iCARs provided herein includes a transmembrane domain from a endogenous polypeptide selected from an activating NK cell receptor, an immunoglobulin protein, B7-H3, BAFFR, BLAME (SLAMF8), BTLA, CD100 (SEMA4D), CD103, CD137, CD160 (BY55), CD18, CD19, CD19a, CD2, CD247, CD27, CD276 (B7-H3), CD28, CD29, CD3delta, CD3 epsilon, CD3 gamma, CD3 zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CTLA-4, CRT AM, cytokine receptor, DAP-10, DNAM1 (CD226), Fe gamma receptor, GADS, GITR, HVEM (LIGHTR), IA4, ICAM-1, Ig alpha (CD79a), IL-2R beta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1), an MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecule (a SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6.

In some embodiments, a transmembrane domain for use in CARs and/or iCARs provided herein comprises portions of transmembrane domains present in two or more endogenous proteins, such that the chimeric transmembrane domain retains the ability to fold correctly and span the cell membrane. In some embodiments, CARs and/or iCARs provided herein include a transmembrane domain that differs from a transmembrane domain present in an endogenous protein by one or more amino acids, e.g., 1, 2, 3, 4, 5, 6, 7, 8, 9, 10, or more amino acids. In some embodiments, CARs and/or iCARs provided herein include a transmembrane domain that shares a degree of amino acid sequence identity to a transmembrane domain present in an endogenous protein. For example, a transmembrane domain for use in a CAR and/or iCARs provided herein can share at least 80%, at least 81%, at least 82%, at least 83%, 84%, 85%, 86%, 87%, 88%, 89%, 90%, 91%, 92%, 93%, 94%, 95%, 96%, 97%, 98%, 99% or more sequence identity with a transmembrane domain present in an endogenous protein.

In some embodiments, the intracellular signaling domain for use in CARs provided herein includes an intracellular signaling domain from an endogenous polypeptide selected from an activating NK cell receptor, an immunoglobulin protein, B7-H3, BAFFR, BLAME (SLAMF8), BTLA, CD100 (SEMA4D), CD103, CD137, CD160 (BY55), CD18, CD19, CD19a, CD2, CD247, CD27, CD276 (B7-H3), CD28, CD29, CD3delta, CD3epsilon, CD3gamma, CD3zeta, CD30, CD4, CD40, CD49a, CD49D, CD49f, CD69, CD7, CD84, CD8, CD8alpha, CD8beta, CD96 (Tactile), CD11a, CD11b, CD11c, CD11d, CDS, CEACAM1, CTLA-4, CRTAM, a cytokine receptor, DAP-10, DNAM1 (CD226), Fc gamma receptor, GADS, GITR, HVEM (LIGHTR), IA4, ICAM-1, Ig alpha (CD79a), IL-2Rbeta, IL-2R gamma, IL-7R alpha, inducible T cell costimulator (ICOS), an integrin, ITGA4, ITGA6, ITGAD, ITGAE, ITGAL, ITGAM, ITGAX, ITGB2, ITGB7, ITGB1, KIRDS2, LAT, ligand that specifically binds with CD83, LIGHT, LTBR, Ly9 (CD229), Lyl08, lymphocyte function-associated antigen-1 (LFA-1), a MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecules (SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6, or any combination thereof.

In some embodiments, the intracellular signaling domain for use in iCARs can be derived from an inhibitory receptor. In some embodiments, the intracellular signaling domain for use in iCARs can antagonize T cell activation. In some embodiments, the intracellular signaling domain for use in iCARs provided herein includes an intracellular signaling domain from an endogenous polypeptide selected from CTLA-4, PD-1, LAG3 HAVCR2 (TIM3), KIR2DL2, LILRB1, TIGIT, CEACAM1, CSF1R, CDS, CD96, CD22, LAIR, 2B4, BTLA, CD3-zeta, or any combination thereof.

In some embodiments, an intracellular signaling domain for use in CARs and/or iCARs provided herein comprises portions of intracellular signaling domains present in two or more endogenous proteins, such that the chimeric intracellular signaling domain retains the ability to fold correctly and mediate signaling (an activating in the case of a CAR, and an inhibiting signal in the case of an iCAR). In some embodiments, CARs and/or iCARs provided herein include an intracellular signaling domain that differs from an intracellular signaling domain present in an endogenous protein by one or more amino acids, e.g., 1, 2, 3, 4, 5, 6, 7, 8, 9, 10, or more amino acids. In some embodiments, CARs and/or iCARs provided herein include an intracellular signaling domain that shares a degree of amino acid sequence identity to an intracellular signaling domain present in an endogenous protein. For example, an intracellular signaling domain for use in a CAR and/or iCARs provided herein can share at least 80%, at least 81%, at least 82%, at least 83%, 84%, 85%, 86%, 87%, 88%, 89%, 90%, 91%, 92%, 93%, 94%, 95%, 96%, 97%, 98%, 99% or more sequence identity with a an intracellular signaling domain present in an endogenous protein.

In some embodiments, CARs provided herein can include a co-stimulatory domain. In some embodiments, CARs provided herein can include two or more co-stimulatory domains.

For example, a CAR can include a co-stimulatory domain that is present in an endogenous polypeptide. Non-limiting examples of polypeptides having co-stimulatory domains that are suitable to include in engineered immune receptors provided herein include 4-1BB (CD137), CD28, CD2, CD4, OX40, ICOS, BTLA, CD27, CD30, GITR, and HVEM, and CD8. In some embodiments, a co-stimulatory domain for use in CARs provided herein comprises portions of co-stimulatory domains present in two or more endogenous proteins, such that the chimeric co-stimulatory domain retains the ability to fold correctly and enhance signaling. In some embodiments, CARs provided herein include a co-stimulatory domain that differs from a co-stimulatory domain present in an endogenous protein by one or more amino acids, e.g., 1, 2, 3, 4, 5, 6, 7, 8, 9, 10, or more amino acids. In some embodiments, CARs provided herein include a co-stimulatory domain that shares a degree of amino acid sequence identity to a co-stimulatory domain present in an endogenous protein. For example, a co-stimulatory domain for use in a CAR provided herein can share at least 80%, at least 81%, at least 82%, at least 83%, 84%, 85%, 86%, 87%, 88%, 89%, 90%, 91%, 92%, 93%, 94%, 95%, 96%, 97%, 98%, 99% or more sequence identity with a co-stimulatory domain present in an endogenous protein.

### Engineered Immune Cells Targeting Loss of Heterozygous Alleles

Clonal loss of heterozygosity (LOH) is a widespread and irreversible genetic alteration that is specific to cancer cells. As used herein, “loss of heterozygosity” refers to a cross chromosomal event that results in a loss of an entire gene and the surrounding chromosomal region. In some embodiments, an LOH event can result in loss of expression of a cell surface antigen that was encoded by the lost gene. In some embodiments, an LOH event can be therapeutically targeted by “inverting” the loss of an allele in cancer cells into an activating signal. In some embodiments, engineered immune cells (e.g., CAR T cells) incorporating NOT-gate logic can be used to target counter-expressed antigens resulting from LOH events in cancer. In some embodiments, engineered immune cells (e.g., CAR T cells) incorporating NIMPLY logic gate can be used to target counter-expressed antigens resulting from LOH events in cancer. Although NOT-gate logic is incorporated and more generally understandable, the full circuit is more accurately described as a NIMPLY logic gate (https://en.wikipedia.org/wiki/NIMPLY_gate). For example, the NIMPLY logic gate can include a chimeric antigen receptor (CAR) targeting an antigen expressed from an allele of human leukocyte antigen (HLA) that is retained in the cancer cells; and an inhibitory CAR (iCAR) targeting the antigen expressed from the HLA allele that is lost in the cancer cells. In some embodiments, engineered immune cells incorporating such a NIMPLY logic gate can be activated in a genetically-predictable manner in vitro and in mice to kill relevant cancer cells.

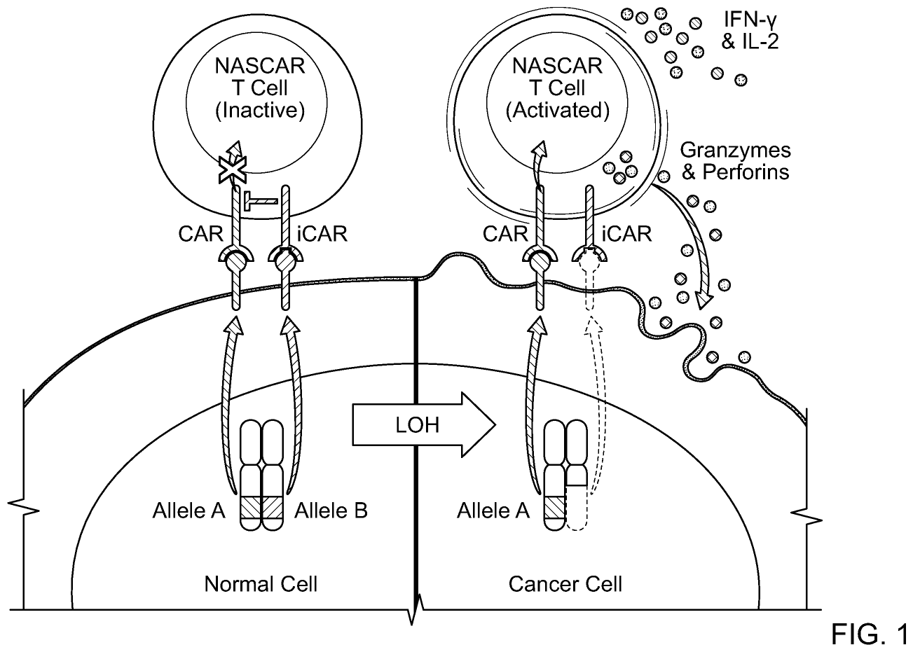

NASCAR (Neoplasm-targeting Allele-Sensing CAR) is a therapeutic approach, which could be extended to LOH of other polymorphic genes that result in altered cell surface antigens in cancers. In some embodiments, NASCAR is a platform including a pair of chimeric antigen receptors (CARs) for detecting and targeting LOH events in cancer cells. In some embodiments, a CAR and an inhibitory CAR (iCAR) are introduced into an immune cell, wherein the CAR and iCAR target two different antigens. In some embodiments, the two different antigens are expressed from two alleles of a same gene. In some embodiments, NASCARs activate the immune cells to kill cells expressing only the CAR antigen, while inactivating the immune cells when cells harbor both the CAR and iCAR antigens. In some embodiments, an immune cell using NASCAR can be applied to an LOH event in cancer by targeting the iCAR to the allele lost through LOH while targeting the CAR to the retained allele (FIG. 1).

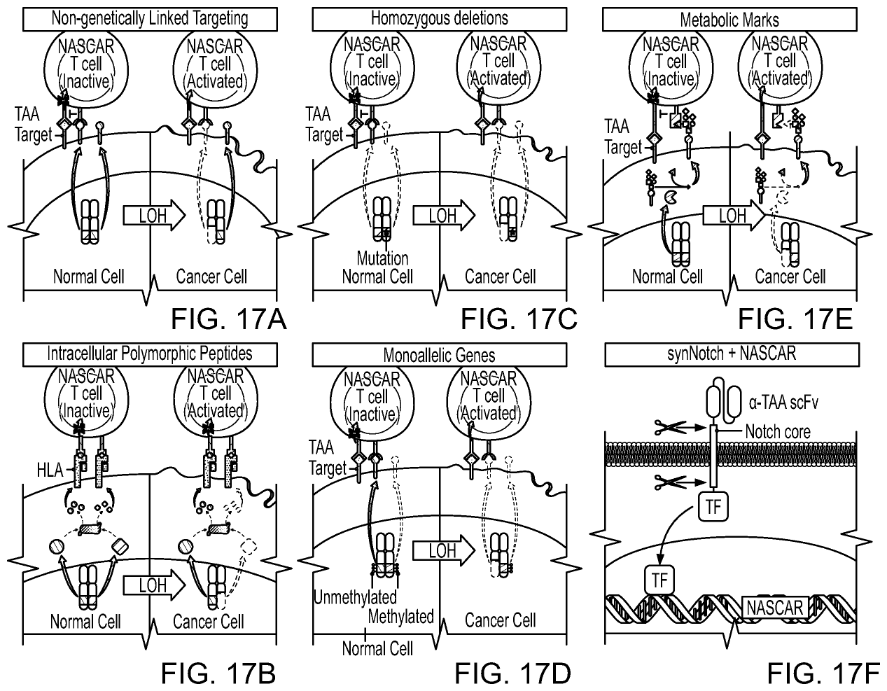

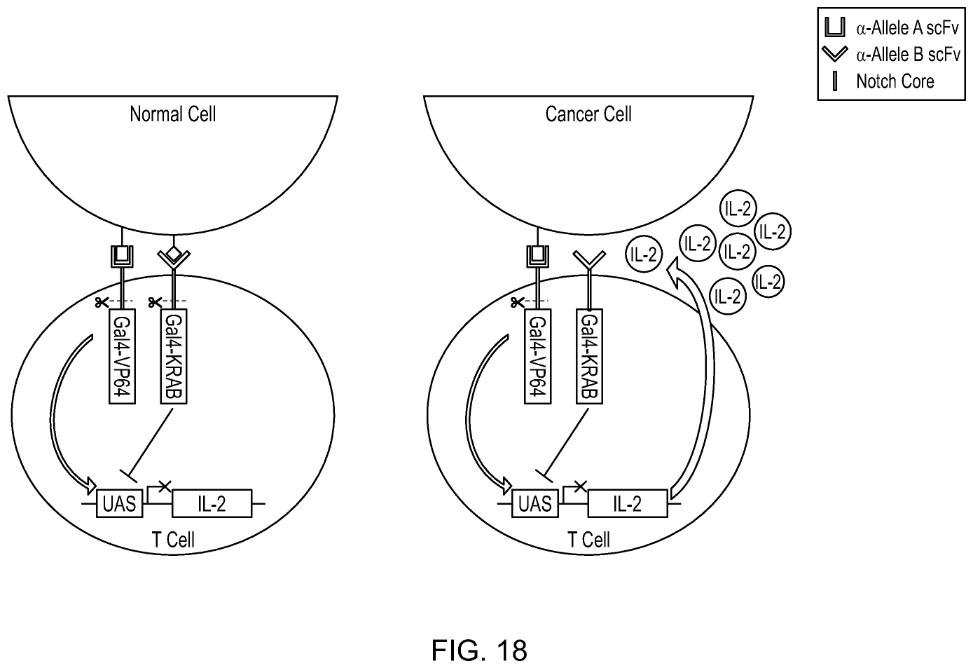

In some embodiments, the CAR and inhibitory CAR (iCAR) target polymorphic forms of the same molecule (e.g., antigen, epitope) (FIG. 1). In some embodiments, the polymorphic forms of the same molecule are co-expressed on a cell. In some embodiments, the CAR and iCAR target different, non-genetically linked molecules (FIG. 17A). In some embodiments, the CAR targets an antigen that is not genetically linked to an allele lost in a LOH event. For example, the CAR antigen can be a tumor-associated antigen (TAA) while the iCAR antigen can be an antigen expressed from a polymorphic allele that is lost in a cancer cell but present on a normal cell. In some embodiments, the antigens targeted by NASCAR do not reside on the cell surface. In some embodiments, a target antigen can be presented on the cell surface by an HLA molecule (FIG. 17B). In some embodiments, NASCAR can target the loss of a marker that is lost in a LOH event due to a genetic deletion (e.g., homozygous deletions) in the retained allele (FIG. 17C). In some embodiments, NASCAR can target the loss of normal monoallelic expression (e.g., epigenetic imprinting or allelic expression) (FIG. 17D). In some embodiments, NASCAR can target the loss of metabolic marks (FIG. 17E). In some embodiments, NASCAR can conditionally express a CAR and iCAR (FIG. 17F). For example, an immune cell with NASCAR can include a synthetic Notch (synNotch) receptor, wherein when the synNotch receptor binds its target antigen (e.g., a tumor-associated antigen) the CAR and iCAR are conditionally expressed on the immune cell (FIG. 18).

Engineered Immune Cells with Two Antigen-Binding Domains

In some embodiments, an immune cell provided herein includes a chimeric antigen receptor (CAR) and an inhibitory chimeric antigen receptor (iCAR) (e.g., NASCAR). In some embodiments, the CAR includes an extracellular antigen binding domain that binds specifically to a first epitope, and the iCAR includes an extracellular antigen binding domain that binds specifically to a second epitope. In some embodiments, the immune cell is activated when the immune cell binds to the first epitope and does not bind to the second epitope, and the immune cell is inactivated when the immune cell binds to the first and second epitopes.

In some embodiments, the first epitope is expressed from a first allele and the second epitope is expressed from a second allele, wherein the first allele and the second allele are from a same gene of a target cell. In some embodiments, the first epitope, the second epitope, or both is expressed from a human leukocyte antigen (HLA) gene. In some embodiments, the first epitope is a tumor-associated antigen (TAA). In some embodiments, the second epitope is expressed from a polymorphic allele that is lost in a cancer cell but present in a normal cell. In some embodiments, the first epitope, the second epitope, or both is present on a surface of a target cell. In some embodiments, the first epitope, the second epitope, or both is present in an extracellular domain of a cell surface protein. In some embodiments, the first epitope, the second epitope, or both is present in an intracellular protein that is presented on the surface of a cell by an HLA molecule.

In some embodiments, the CAR is capable of specifically binding a first epitope (e.g., a tumor associated antigen), and the iCAR is capable of specifically binding a second epitope (e.g., an antigen expressed from a polymorphic allele that is lost in a cancer cell but present in a normal cell). For example, the CAR can bind a first epitope and the iCAR can bind a second epitope on a target cell, wherein the first and second epitopes are expressed from alleles of a same gene, and wherein the iCAR inhibits the activation of the immune cell. However, when the target cell includes a genetic abnormality (e.g., loss of heterozygosity (LOH)), the CAR can bind to the first epitope but the iCAR cannot bind to the second epitope because the allele expressing the second epitope is lost in the target cell, thereby activating the immune cell.

In some embodiments, specific binding of only a first epitope to the CAR of the immune cell results in an increase in activation of the immune cell as compared to when a first epitope and a second epitope are both specifically bound to the immune cell and the iCAR inhibits the activation of the immune cell. In some embodiments, activation of an immune cell expressing a CAR and/or an iCAR is measured. Any of a variety of assays to measure immune cell activation (e.g., any of the assays disclosed herein) can be used. Non-limiting examples include cytolytic or cytotoxic assays (e.g., lactate dehydrogenase (LDH) release), cytokine release assays (e.g., cytokine release assays measuring release of interleukin-2 (IL-2) and interferon-gamma (IFN-gamma)), and measuring F-actin accumulation at the immune synapse. See, e.g., Stenken et al, “Bioanalytical Chemistry of Cytokines-A Review”, Anal Chim Acta 2015; Turner et al, “Cytokines and chemokines: At the crossroads of cell signalling and inflammatory disease”, Biochimica et Biophysica Acta Molecular Cell Research 2014; Clay et al, “Assays for monitoring cellular immune responses to active immunotherapy of cancer”, Clin Cancer Res 2001; Wonderlich et al, “UNIT 3.11 Induction and Measurement of Cytotoxic T Lymphocyte Activity”, Current Protocols in Immunology 2006, each of which is incorporated herein by reference in its entirety. Those of ordinary skill in the art will be aware of other suitable assays to activation of immune cells.

In some embodiments, an immune cell expressing a CAR and an iCAR can be cytotoxic or cytostatic to a target cell in vitro or in vivo (e.g., a mammalian cancer cell). For example when only a first epitope expressed on the surface of a target cell specifically binds to a CAR, the immune cell is cytotoxic or cytostatic to a target cell. In some embodiments, when a first epitope and a second epitope are both specifically bound to the immune cell and the iCAR inhibits the activation of the immune cell, the immune cell can have reduced cytotoxicity or cytostatic activity toward a non-target cell (e.g., a non-cancerous mammalian cell).

In some embodiments, the CAR and the iCAR are conditionally expressed in the immune cell. In some embodiments, the CAR and the iCAR are expressed in the immune cell in a tumor microenvironment. In some embodiments, the immune cell further comprises a synNotch receptor, wherein the synNotch receptor activates the CAR and the iCAR expression when the immune cell is in the tumor microenvironment. See, e.g., Kingwell. K., Nat Rev Drug Discov (2016) 15, 819, the disclosure of which is incorporated herein by reference in its entirety.

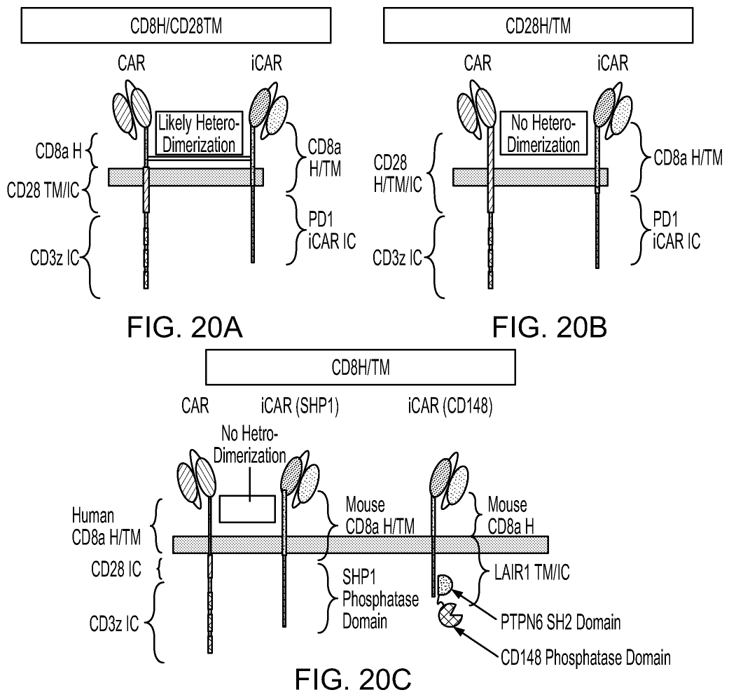

For example, the CAR construct can include an scfv, CD8alpha hinge domain, CD28 transmembrane and intracellular domain, and a CD3zeta intracellular domain and the iCAR can include an scfv, CD8alpha hinge and transmembrane domain, and a PD-1 intracellular domain (FIG. 20A). In some embodiments, since both CAR and iCAR constructs contain CD8alpha hinge domains, there can be heterodimerization on the T cell surface. For example, the CAR construct can include an scfv, CD28 hinge/transmembrane/intracellular domains, and a CD3zeta intracellular domain and the iCAR can include an scfv, CD8alpha hinge and transmembrane domain, and a PD-1 intracellular domain (FIG. 20B). In some embodiments, since CAR and iCAR constructs contain different hinges and transmembrane domains, there is may be no heterodimerization. For example, the CAR construct can include an scfv, human CD8alpha hinge and transmembrane domains, CD28 intracellular domain, and a CD3zeta intracellular domain and the two iCARs displayed both have an scfv attached to a mouse CD8alpha hinge domain (FIG. 20C) (to prevent heterodimerization). In some embodiments, one iCAR variant includes a mouse CD8alpha transmembrane domain connected to a SUP-1 domain. In some embodiments, the other iCAR variant includes a LAIR1 transmembrane and intracellular domain and a separate chimeric molecule in which a PTPN6 SH2 domain is linked to a CD148 phosphatase domain.

Expression of a CAR and iCAR in an Immune Cell

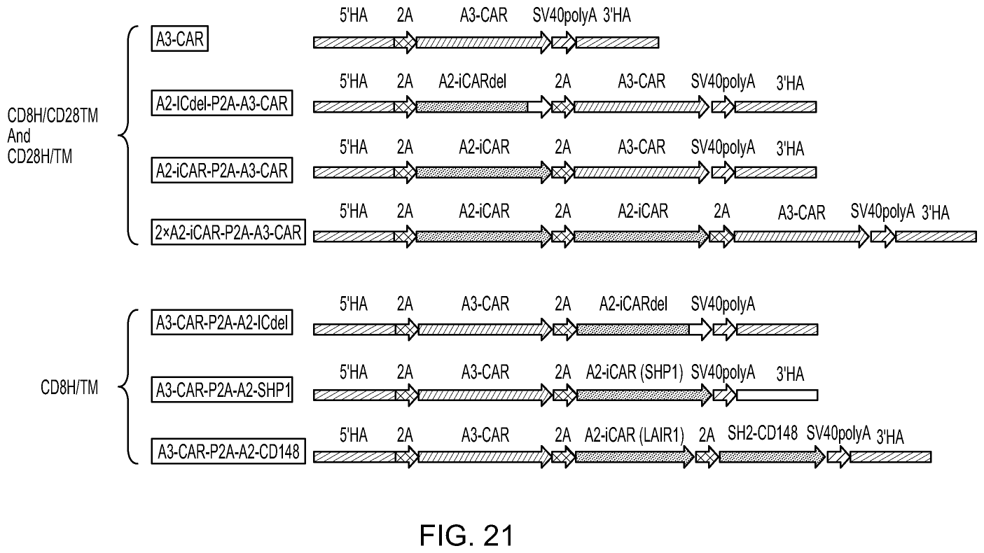

Methods of generating an immune cell that expresses a CAR and an iCAR (e.g., any of the CARs and/or iCARs described herein) include: introducing into a cell a nucleic acid sequence encoding the CAR and iCAR to produce a recombinant cell; and culturing the recombinant cell under conditions sufficient for the expression of the CAR and iCAR. In some embodiments, the introducing step includes introducing into a cell expression vectors including a sequence encoding the CAR and iCAR to produce a recombinant cell. For example, CAR and iCAR constructs can be designed to be introduced into immune cells (e.g., T cells) as dsDNA templates via homology directed repair at the B2M locus after a CRISPR Cas9 induced double stranded DNA break. In some embodiments, major components can include, but are not limited 30 to, homology arms, 2A sequences, CAR, iCAR, and polyA terminators (FIG. 21).

A CAR and/or iCAR described herein can be produced by any cell, e.g., a eukaryotic cell or a prokaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. As used herein, the term “prokaryotic cell” refers to a cell that does not have a distinct, membrane-bound nucleus. In some embodiments, the prokaryotic cell is a bacterial cell.

In some embodiments, a CAR and iCAR can be expressed in a cell selected from the group consisting of: a CD8+ T cell and a CD4+ T cell. In some embodiments, a CAR and iCAR are expressed in a cell (e.g., an immune cell) that can be administered to a subject, which cell is autologous to a subject. For example, an immune cell can be isolated from a subject, transfected with an expression vector or vectors encoding the CAR and iCAR, and subsequently administered back to the subject. In some embodiments, a CAR and iCAR are expressed in a cell (e.g., an immune cell) that can be administered to a subject, which cell is allogeneic to a subject. For example, an immune cell can be isolated from a donor (e.g., another human), transfected with an expression vector or vectors encoding the CAR and iCAR, and subsequently administered to the subject. In some embodiments, a CAR and iCAR are expressed in an induced pluripotent stem cell (iPSC), which iPSC is then administered to the subject. For example, a cell (e.g., an adult cell) can be isolated from a subject, induced to become a pluripotent stem cell, further induced to become an immune cell (e.g., a CD8+ T cell or a CD4+ T cell), wherein a CAR and iCAR are expressed in the cell (e.g., via any of the methods disclosed herein). In some embodiments, a vector or vectors encoding a CAR and iCAR can be introduced into an iPSC prior to its induction to become an immune cell. In some embodiments, a vector or vectors encoding a CAR and iCAR can be introduced into an immune cell after it has been induced to differentiate from an iPSC into an immune cell.

Methods of culturing cells are well known in the art. Cells can be maintained in vitro under conditions that favor proliferation, differentiation, and growth. Briefly, cells can be cultured by contacting a cell (e.g., any cell) with a cell culture medium that includes the necessary growth factors and supplements to support cell viability and growth.

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalection, hydrodynamic delivery, magnetofection, viral transduction (e.g., adenoviral and lentiviral transduction), and nanoparticle transfection.

In some embodiments, expression of a CAR and iCAR in a cell is regulated by one or more mechanisms. For example, a nucleic acid comprising a nucleotide sequence encoding a CAR can be operably linked to a promoter, an enhancer, or both. Also, a nucleic acid comprising a nucleotide sequence encoding an iCAR can be operably linked to a promoter, an enhancer, or both. Suitable promoters (e.g., inducible promoters) and enhancers for regulating expression of vectors encoding polypeptides in cells are known to those of ordinary skill in the art.

### Therapeutic Applications

In some embodiments, provided herein are methods of treating a subject having a disease, wherein the method includes administering to the subject a composition that includes or delivers an immune cell expressing a CAR and an iCAR. In some embodiments, the disease is a cancer. In some embodiments, the disease is a precancerous pathology with LOH.

Cancer can refer to a broad group of diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. Cancer or cancer tissue may include a tumor.

Cancers suitable for treatment by a method of the present disclosure can include, but are not limited to, bladder cancer, breast cancer, cervical cancer, colon cancer, endometrial cancer, esophageal cancer, fallopian tube cancer, gall bladder cancer, gastrointestinal cancer, head and neck cancer, hematological cancer, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma, and prostate cancer. Non-limiting examples of cancer include: acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms' tumor.

In some embodiments, effective doses can vary depending on the risk and/or the severity of the cancer, the route of administration, the age and general health condition of the subject, excipient usage, the possibility of co-usage with other therapeutic treatments such as use of other agents, and the judgment of the treating physician. An effective amount of immune cells expressing a CAR and an iCAR can be any amount that treats a cancer present within the subject without producing significant toxicity to the subject. If a particular subject fails to respond to a particular amount, then the amount of one or more molecules including one or more antigen-binding domains (e.g., scFvs) that can bind to a modified peptide described herein can be increased (e.g., by two-fold, three-fold, four-fold, or more). After receiving this higher amount, the subject can be monitored for both responsiveness to the treatment and toxicity symptoms, and adjustments made accordingly. The effective amount can remain constant or can be adjusted as a sliding scale or variable dose depending on the subject's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

In some embodiments, the disease is a premalignant pathologic condition or similar precancerous condition including but not limited to myelodysplastic syndromes, acquired aplastic anemia, Fanconi anemia, paroxysmal nocturnal hemoglobinuria (PNH), 5q-syndrome and any condition characterized by pathogenic cells with clonal LOH. In some embodiments, the disease is a hematologic malignancy (e.g, myelodysplastic syndrome (MDS), acute myeloid leukemia (AML), acute lymphocytic leukemia (ALL)). In some embodiments, the immune cells expressing a CAR and an iCAR can target LOH which is present in the hematologic malignancy that relapse after matched, mis-matched and haploidentical blood or marrow transplantation. In some embodiments, the immune cells expressing a CAR and an iCAR can target LOH which are present in cancer cells and cause relapse after targeted CAR T cell therapy or bispecific antibody therapy directed against tumor associated antigens (e.g., CD19 or NY-ESO-1).

In some embodiments, the frequency of administration of immune cells expressing a CAR and an iCAR provided herein can be any frequency that effectively treats a mammal having a cancer without producing significant toxicity to the mammal. For example, the frequency of administration of immune cells expressing a CAR and an iCAR can be from about two to about three times a week to about two to about three times a year. In some cases, a subject having cancer can receive a single administration of immune cells expressing a CAR and an iCAR. The frequency of administration of immune cells expressing a CAR and an iCAR can remain constant or can be variable during the duration of treatment. A course of treatment with immune cells expressing a CAR and an iCAR can include rest periods. For example, immune cells expressing a CAR and an iCAR can be administered every other month over a two-year period followed by a six-month rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

In some embodiments, an effective duration for administering immune cells expressing a CAR and an iCAR can be any duration that effectively treats a cancer present within the subject without producing significant toxicity to the subject. In some cases, the effective duration can vary from several months to several years. In general, the effective duration for treating a subject having a cancer can range in duration from about one or two months to five or more years. Multiple factors can influence the actual effective duration used for a particular treatment. For example, an effective duration can vary with the frequency of administration, effective amount, use of multiple treatment agents, route of administration, and severity of the condition being treated.

In certain instances, a cancer within a subject can be monitored to evaluate the effectiveness of the cancer treatment. Any appropriate method can be used to determine whether or not a subject having cancer is treated. For example, imaging techniques or laboratory assays can be used to assess the number of cancer cells and/or the size of a tumor present within a subject. For example, imaging techniques or laboratory assays can be used to assess the location of cancer cells and/or a tumor present within a subject.

In some embodiments, immune cells expressing a CAR and an iCAR can be administered to a subject having a cancer as a combination therapy with one or more additional cancer treatments. A cancer treatment can include any appropriate cancer treatments. For example, a cancer treatment can include surgery. For example, a cancer treatment can include radiation therapy. For example, a cancer treatment can include administration of one or more therapeutic agents (e.g., one or more anti-cancer agents). In some cases, an anti-cancer agent can be an immunotherapy (e.g., a checkpoint inhibitor). In some embodiments, the subject has previously been administered one or more additional anticancer therapies selected from the group consisting of ionizing radiation, a chemotherapeutic agent, a therapeutic antibody, or a checkpoint inhibitor. In some embodiments, the subject will be administered one or more additional anticancer therapies selected from the group consisting of ionizing radiation, a chemotherapeutic agent, a therapeutic antibody, or a checkpoint inhibitor.

### Pharmaceutical Compositions

In some embodiments, the present disclosure provides pharmaceutical compositions that include an immune cell comprising a CAR and an iCAR, and a pharmaceutically acceptable carrier. In some embodiments, a pharmaceutical composition can include a buffer, a diluent, solubilizer, emulsifier, preservative, adjuvant, an excipient, or any combination thereof. In some embodiments, a composition, if desired, can also contain one or more additional therapeutically active substances.

In some embodiments, immune cells of the present disclosure are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

In some embodiments, compositions are formulated for parenteral administration. For example, a pharmaceutical composition provided herein may be provided in a sterile injectable form (e.g., a form that is suitable for subcutaneous injection or intravenous infusion). For example, in some embodiments, a pharmaceutical compositions is provided in a liquid dosage form that is suitable for injection. In some embodiments, a pharmaceutical composition is provided as powders (e.g., lyophilized and/or sterilized), optionally under vacuum, which can be reconstituted with an aqueous diluent (e.g., water, buffer, salt solution, etc.) prior to injection. In some embodiments, a pharmaceutical composition is diluted and/or reconstituted in water, sodium chloride solution, sodium acetate solution, benzyl alcohol solution, phosphate buffered saline, etc. In some embodiments, a powder should be mixed gently with the aqueous diluent (e.g., not shaken).

In some embodiments, an immune cell including a CAR and an iCAR of the present disclosure is formulated with a pharmaceutically acceptable parenteral vehicle. Examples of such vehicles are water, saline, Ringer's solution, dextrose solution, and 1-10% human serum albumin. Liposomes and nonaqueous vehicles such as fixed oils can also be used. A vehicle or lyophilized powder can contain additives that maintain isotonicity (e.g., sodium chloride, mannitol) and chemical stability (e.g., buffers and preservatives). In some embodiments, a formulation is sterilized by known or suitable techniques. A pharmaceutical composition may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, M D, 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

### EXAMPLES

The disclosure is further described in the following examples, which do not limit the scope of the disclosure described in the claims.

### Example 1—Identification of HLA-A Allele-Specific Detection Moieties

Loss of heterozygosity (LOH) of the short arm of chromosome 6 (6p)—where the human leukocyte antigen (HLA) gene complex resides—has been reported in as many as 24%, 40%, 41%, 41%, and 50% of breast, colon, lung, brain (glioblastoma), and pancreatic cancers, respectively (Table 1). As HLA-A*02:01 and HLA-A*03:01 (henceforth referred to as “A2” and “A3”) are among the most common HLA-A alleles represented in the human population, reagents targeting these two alleles were developed to maximize the patient population that could conceivably benefit from an LOH-directed therapy.

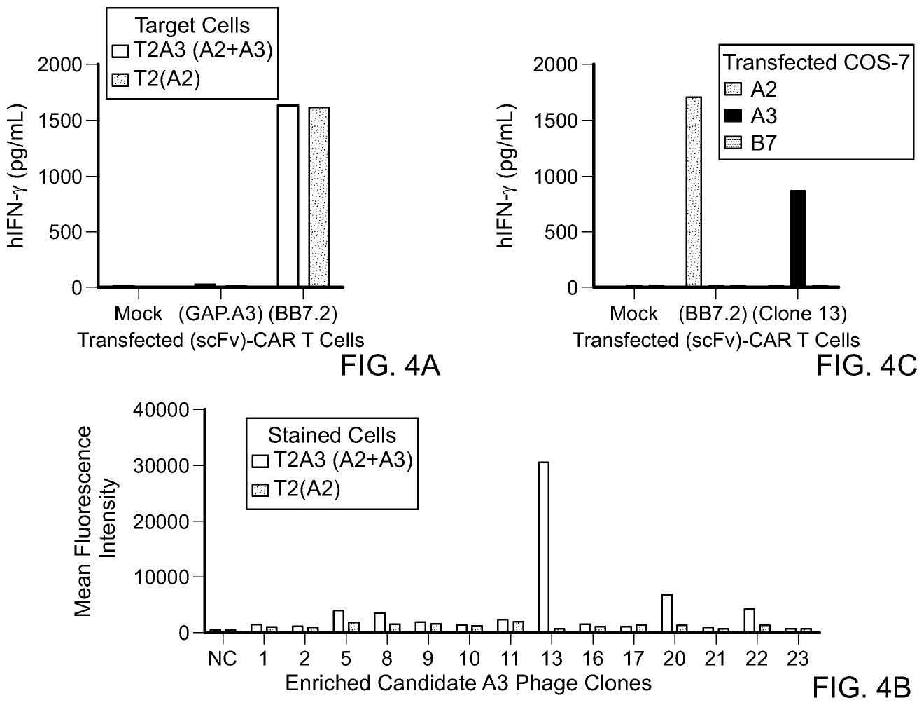

HLA allele-specific antibodies were used for A2 and A3, clones BB7.2 and clone 13, respectively. A new A3-specific scFv, clone 13, was developed, wherein an scFv phage display library, with an estimated complexity of 3.6×10¹⁰, was screened for binders that could selectively target A3 but not other HLA-A alleles. Positive selection was conducted with a different A3 peptide-HLA (pHLA) monomer during each round of panning so as to enforce specificity to the HLA molecule itself and not the associated peptide, while negative selection was performed with a cocktail of non-A3 pHLA monomers. Enriched candidate phage clones were amplified and assessed for their ability to bind to cells with either A2 alone (T2 cells) or A2 and A3 (T2A3 cells) via flow cytometry. Clone 13 was chosen for its strong ability to bind to T2A3 cells but not to T2 cells relative to the other phage clones tested (FIG. 4B). It was then confirmed whether this selected A3-specific scFv could be functionally grafted onto a CAR molecule while maintaining specificity. As expected, Clone 13- and BB7.2-engineered CAR T cells were only activated when exposed to COS-7 target cells transfected with A3 or A2, respectively, as assessed by IFN-γ release (FIG. 4C).

TABLE 1

| Cancer Type | Reported 6p Arm Loss | Reference |
|---|---|---|
| Bladder | 35% | (96) |
| Breast | 24% | (98) |
| | 27% | (126) |
| Cervical | 68% | (91) |
| Colon | 13.8% | (89) |
| | 25% | (19) |
| | 40% | (96) |
| Glioblastoma | 41.4% | (97) |
| Laryngeal | 17.6% | (89) |
| | 53% | (96) |
| Lung | 40% | (93) |
| | 41% | (95) |
| Melanoma | 15.3% | (89) |
| | 23% | (96) |
| Ovarian | 28% | (127) |
| Pancreas | 50% | (94) |
| Renal | 6% | (96) |

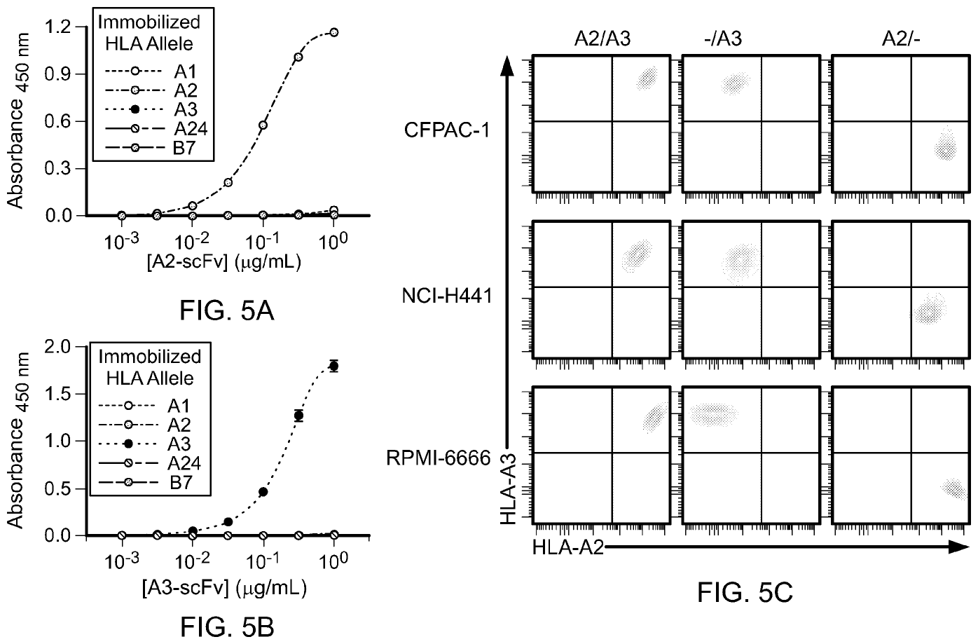

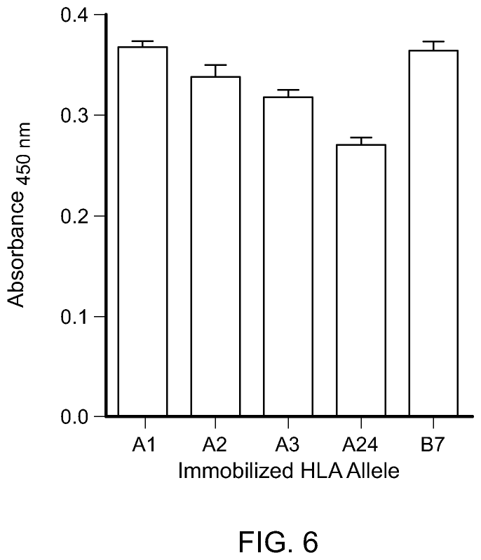

The specificities of the targeting moieties were further evaluated by titration enzyme-linked immunosorbent assay (ELISA) using recombinantly-expressed scFvs. Bacterial expression vectors for the A2- and A3-specific scFvs were generated based on the BB7.2 and Clone 13 sequences in the pAP-III₆ backbone, respectively. pHLA monomers from each of the four HLA-A superfamilies were tested, as was a common HLA-B allele (HLA-B*07:02). As expected, the A2 and A3 scFvs specifically bound to their cognate HLA allele but did not bind to any of the other alleles tested (FIGS. 5A-5B). pHLA monomer complexes were confirmed to be comparably folded, as verified by ELISA via detection with W6/32, a pan-HLA class I antibody (FIG. 6).

### Example 2—Generation of HLA LOH Isogenic Cell Line Models

Next, CRISPR technology was employed to generate isogenic knockout (KO) clones from cancer cell lines expressing endogenous A2 and A3 alleles. Three cell lines of differing cancer types with varied HLA expression levels were selected—CFPAC-1 (pancreatic), NCI-H441 (lung), RPMI-6666 (Hodgkin lymphoma)—and HLA single-allele KO clones for all three cell lines were obtained (FIG. 5C). The clones showed 100% identity match with their originating parental cell line as assessed by short tandem repeat (STR) profiling (Table 2).

TABLE 2

| Query Name | Top Matches | Match % | TH01 | D5S818 | D13S317 | D7S820 | D16S539 | CSF1PO | vWA | TPOX |
|---|---|---|---|---|---|---|---|---|---|---|
| CFPAC-1 (A2/A3) | CFPAC-1 [CRL-1918] | 100 | 8 | 10, 11 | 12 | 8, 10 | 9, 11 | 10 | 17 | 8 |
| CFPAC-1 (—/A3) | CFPAC-1 [CRL-1918] | 100 | 8 | 10, 11 | 12 | 8, 10 | 9, 11 | 10 | 17 | 8 |
| CFPAC-1 (A2/—) | CFPAC-1 [CRL-1918] | 100 | 8 | 10, 11 | 12 | 8, 10 | 9, 11 | 10 | 17 | 8 |
| NCI-H441 (A2/A3) | NCI-H441 [HTB-174] | 100 | 9.3 | 11, 12 | 9 | 10 | 9, 13 | 11, 12 | 17 | 8, 10 |
| NCI-H441 (—/A3) | NCI-H441 [HTB-174] | 100 | 9.3 | 11, 12 | 9 | 10 | 9, 13 | 11, 12 | 17 | 8, 10 |
| NCI-H441 (A2/—) | NCI-H441 [HTB-174] | 100 | 9.3 | 11, 12 | 9 | 10 | 9, 13 | 11, 12 | 17 | 8, 10 |
| RPMI-6666 LucGFP | RPMI-6666 [CCL-113] | 100 | 6, 9 | 11, 12 | 11 | 9, 10 | 11, 12 | 12, 13 | 14, 18 | 10, 11 |
| (A2/A3) | | | | | | | | | | |
| RPMI-6666 LucGFP | RPMI-6666 [CCL-113] | 100 | 6, 9 | 11, 12 | 11 | 9, 10 | 11, 12 | 12, 13 | 14, 18 | 10, 11 |
| (—/A3) | | | | | | | | | | |
| RPMI-6666 LucGFP | RPMI-6666 [CCL-113] | 100 | 6, 9 | 11, 12 | 11 | 9, 10 | 11, 12 | 12, 13 | 14, 18 | 10, 11 |
| (A2/—) | | | | | | | | | | |

### Example 3—Development and Optimization of LOH Detection

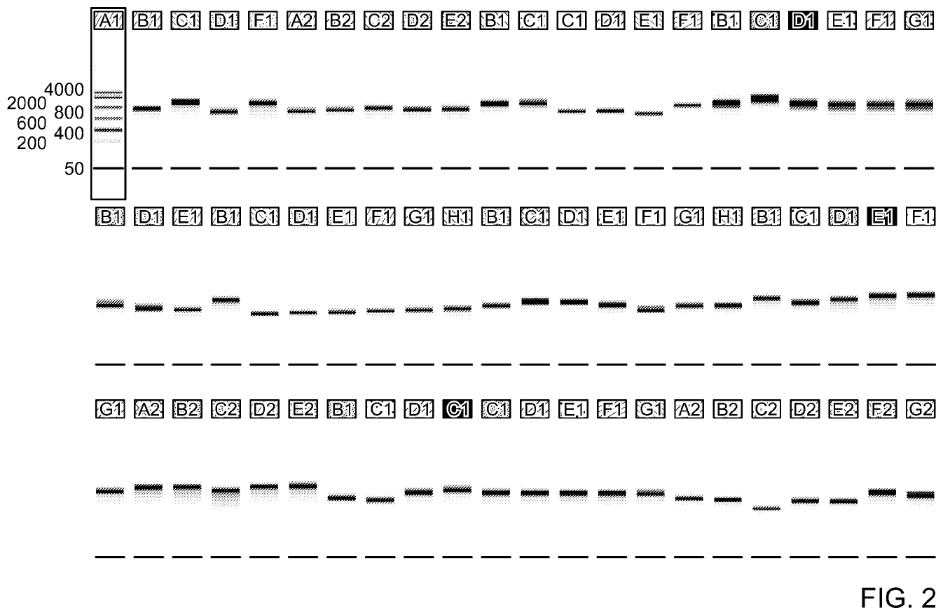

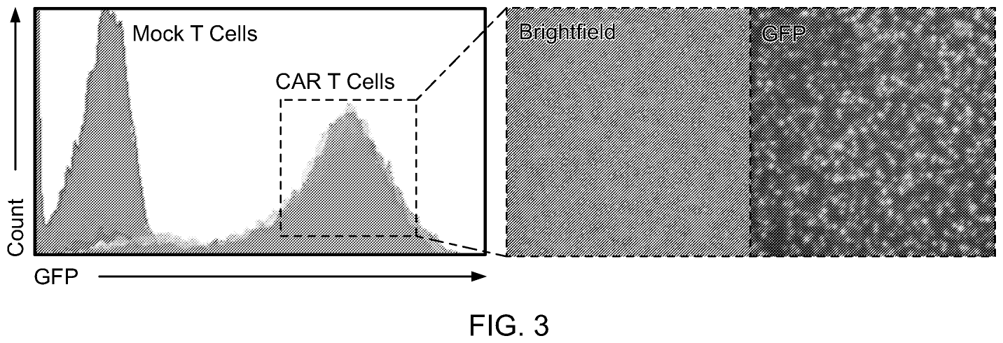

A cell-based therapy platform was chosen due to the innate ability of a cell to integrate a multitude of signals and inputs to drive a coordinated cellular response program. Accordingly, chimeric receptors in T cells were developed, targeting either A2 or A3, which could permit allelic discrimination (i.e. Neoplasm-targeting Allele-Sensing (NASCAR) T cells). An mRNA electroporation-based expression system was initially employed to allow for facile and rapid iteration of candidate constructs. An optimized protocol for primary human T cells was developed, which resulted in virtually all cells expressing the desired proteins with high cell viability following electroporation (FIG. 2, FIG. 3).

To enable LOH detection, the HLA-A allele-specific scFvs were grafted to chimeric receptors with either activating (CAR) or inhibitory (iCAR) signaling domains. To maximize specificity, a systematic optimization of various parameters for each component of the NASCAR targeting platform (i.e. iCAR format, CAR hinge, stoichiometry between CAR and iCAR) was performed.

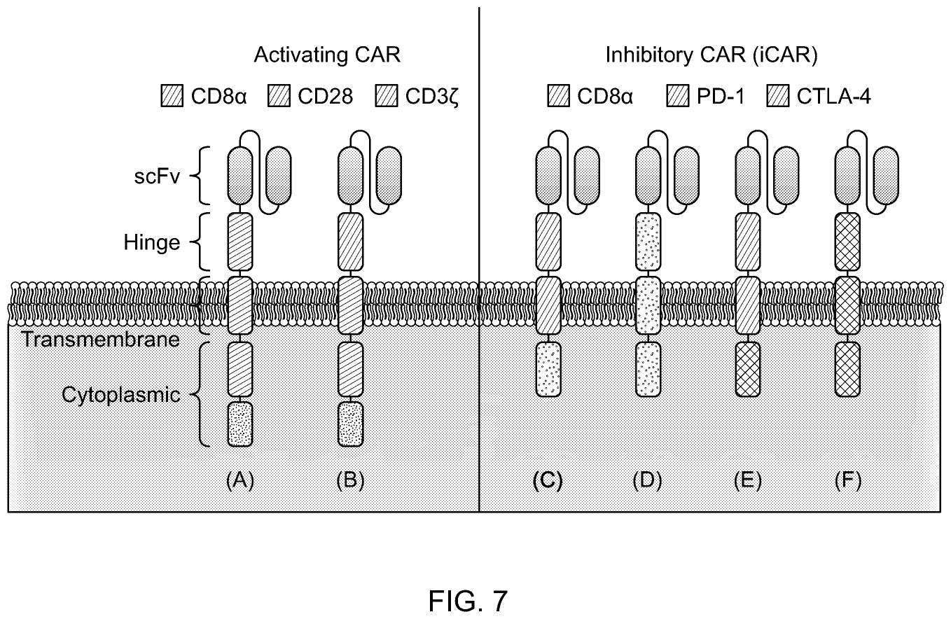

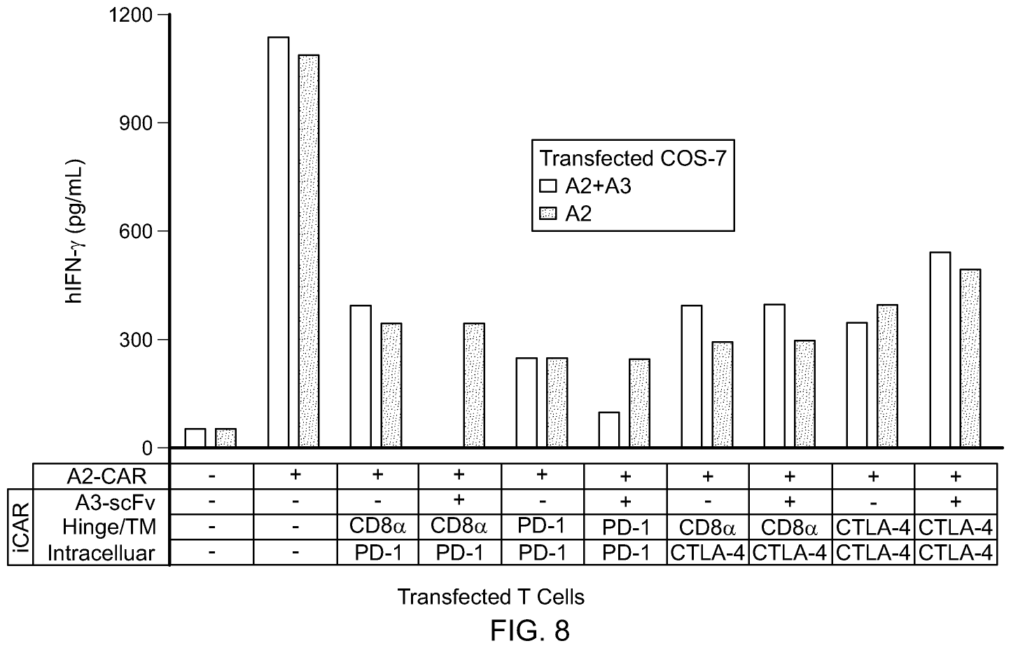

A 2ⁿd generation CAR was employed as the activating construct, comprising of CD28 hinge, transmembrane, and cytoplasmic domains fused to the CD3 (cytoplasmic domain (FIG. 7). Four iCAR constructs incorporating CTLA-4 or PD-1 inhibitory domains with various hinge and transmembrane combinations were screened. For each inhibitory domain, two combinations were tested. Either the CTLA-4 or PD-1 cytoplasmic domain was fused to CD8α hinge and transmembrane domains, or the CTLA-4 or PD-1 cytoplasmic domain was used with each inhibitory receptor's cognate hinge and transmembrane domains (FIG. 7). Control constructs wherein the targeting scFv moiety for each iCAR construct was deleted were also created. To evaluate iCAR performance, T cells co-expressing a CAR in combination with each individual iCAR were co-incubated with A2 or A2+A3-transfected COS-7 cells. Only the PD-1 iCAR constructs resulted in substantial allele-specific inhibition as evidenced by a reduction in IFN-7 secretion towards A2+A3-transfected COS-7 cells relative to A2-transfected cells (FIG. 8). The iCAR construct comprising the PD-1 cytoplasmic domain with CD8α hinge and transmembrane domain exhibited the most potent allele specificity and was therefore selected as the NASCAR inhibitory module for the experiments described below.

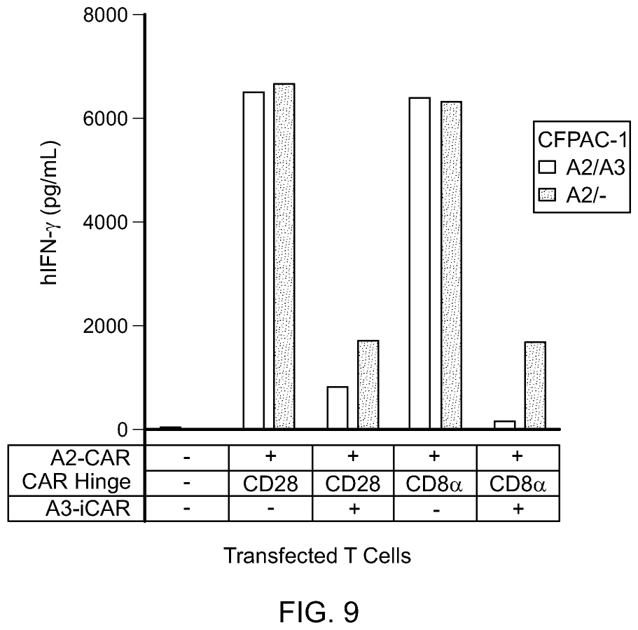

It was postulated that adjusting the CAR hinge domain from CD28 to CD8α would allow for greater iCAR-mediated quenching of proximal CAR signaling by permitting CD8α hinge heterodimerization between CAR and iCAR (FIG. 7). Indeed, iCAR-mediated inhibition of CAR activity was strengthened with a CD8α-hinged CAR when engaged with CFPAC-1 target cells expressing both A2 and A3, as assessed by IFN-7 release (FIG. 9). As the CD8α-hinged CAR conferred an increased window of allele specificity, it was selected as the NASCAR activating module for the experiments described below.

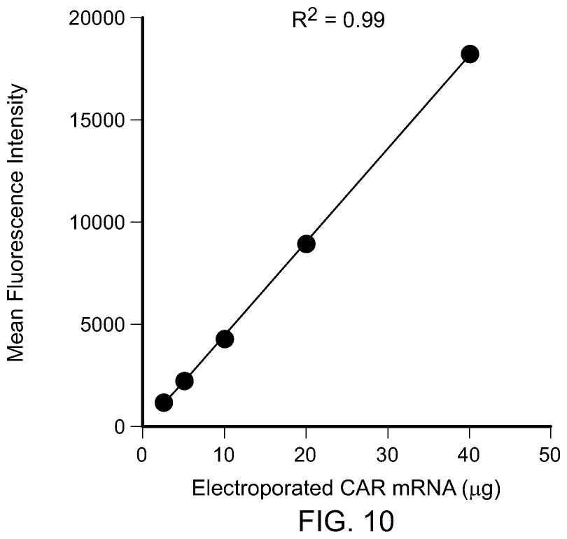

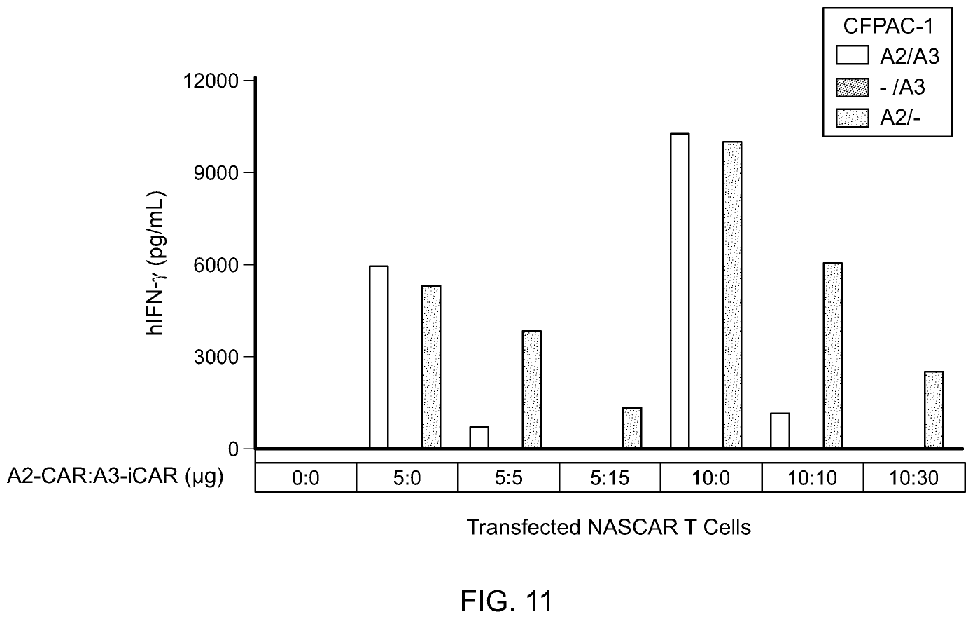

It was observed that the mRNA-based expression system for introducing chimeric receptors into primary human T cells provided a linear correlation between the amount of mRNA electroporated and the corresponding CAR expression level (FIG. 10). Thus, it was explored whether adjusting the stoichiometry between CAR and iCAR could allow for greater specificity to the system. While a 1:1 ratio of CAR:iCAR mRNA displayed considerable allele specificity, there was still significant off-target activation as evidenced by detectable IFN-7 secretion with CFPAC-1 A2/A3 target cells. However, decreasing the CAR:iCAR ratio to 1:3 resulted in pronounced suppression of T-cell activation towards off-target A2/A3 cells (FIG. 11). The 1:3 ratio of CAR:iCAR was therefore selected for the NASCAR experiments described below.

### Example 4—Determination of NASCAR Specificity In Vitro

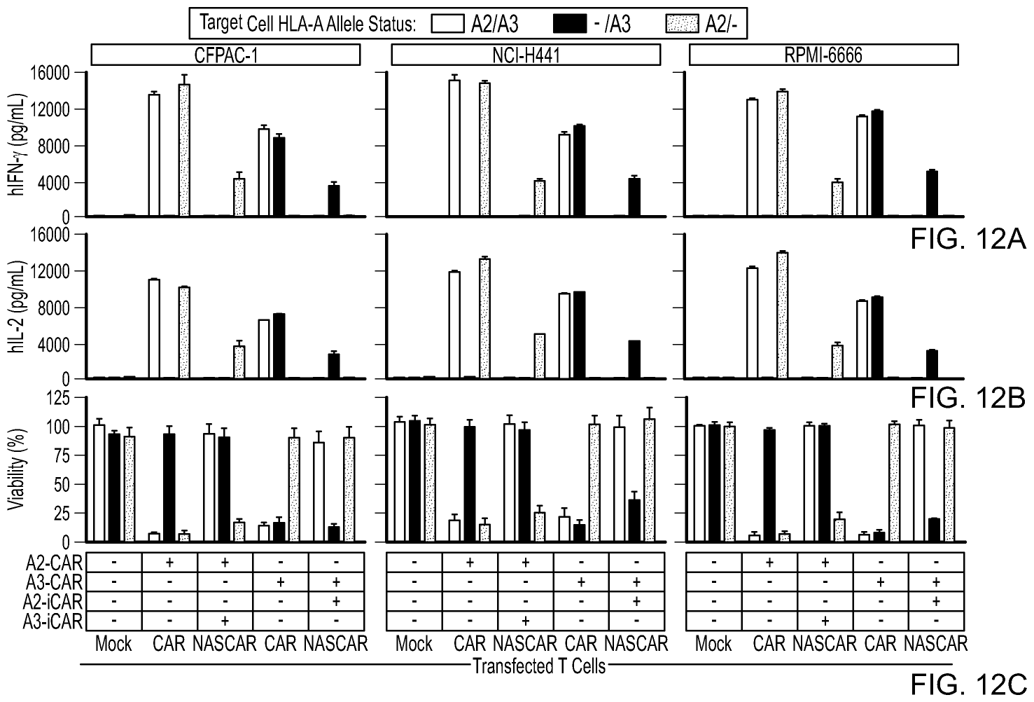

To thoroughly demonstrate the modularity, specificity, and symmetry of the NASCAR approach, both combinations of CAR and iCAR were tested (i.e. A2-CAR+A3-iCAR, A3-CAR+A2-iCAR) against all three sets of isogenic cell lines. T-cell activation, as assessed by IFN-γ and interleukin-2 (IL-2) cytokine release, was remarkably similar across all three cell line backgrounds and revealed the expected allele-specific targeting profiles (FIGS. 12A-12B). Consistently, the degree of cytotoxicity mirrored that of cytokine release (FIG. 12C).

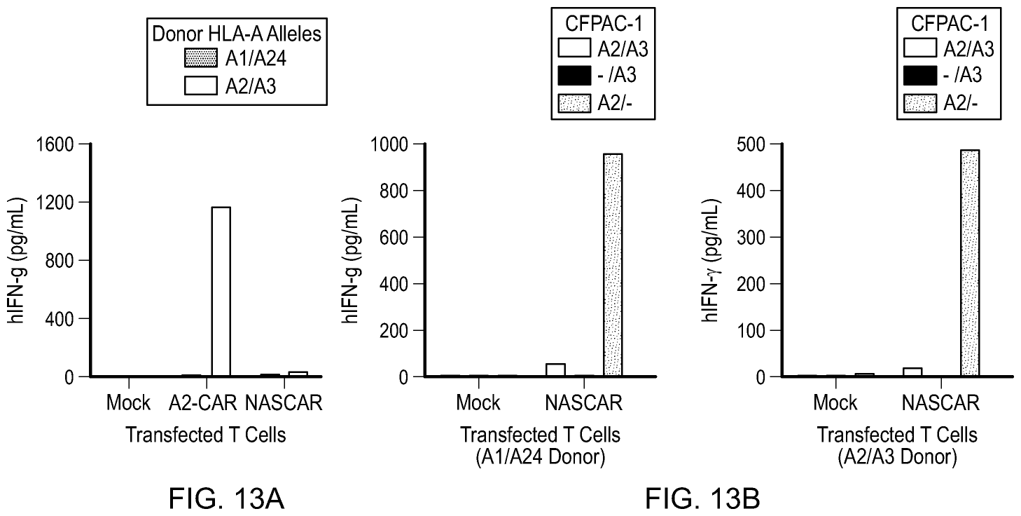

The assays described above employed primary human T cells from A2/A3-negative donors as effector cells. Otherwise, in the control conditions with the CAR but without the iCAR, the effector T cells would commit fratricide due to self-expression of the target antigen. In practice, however, effector T cells would likely be derived from autologous sources that express both A2 and A3. To that end, autoreactivity in model “autologous” antigen-positive (A2/A3) versus model “allogeneic” antigen-negative (A1/A24) effector donor T cells was assayed. While A1/A24 donor T cells elicited no IFN-γ signal as they lacked the activating antigen, introduction of the CAR alone into A2/A3 donor T cells resulted in the expected autoreactivity and fratricide. In contrast, NASCAR expression of the inhibitory module together with the activating module in A2/A3 donor T cells did not result in fratricide (FIG. 13A). Furthermore, NASCAR-engineered A2/A3 donor T cells were as effective in targeting cancer cells with LOH as A1/A24 donor T cells, as assessed by IFN-γ release (FIG. 13B).

### Example 5—Evaluation of NASCAR Antitumor Activity In Vivo

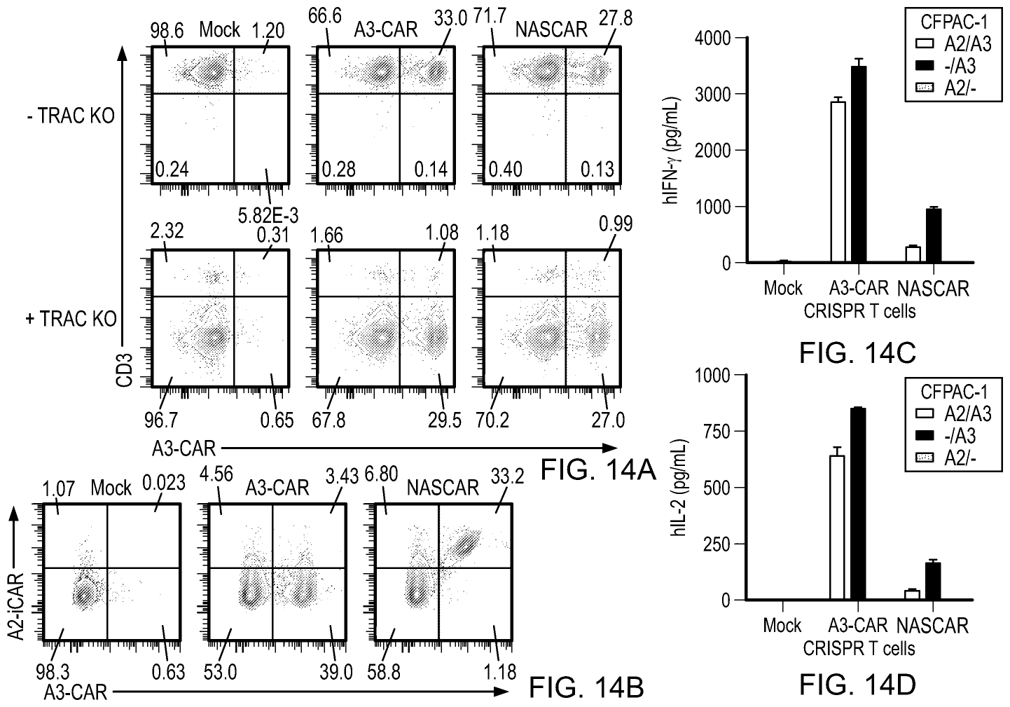

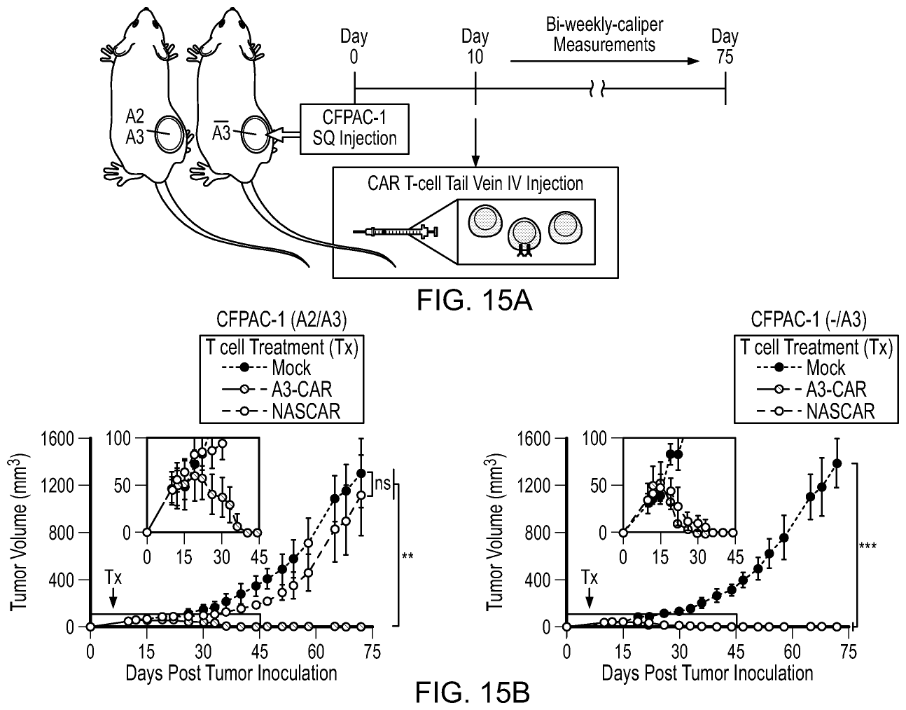

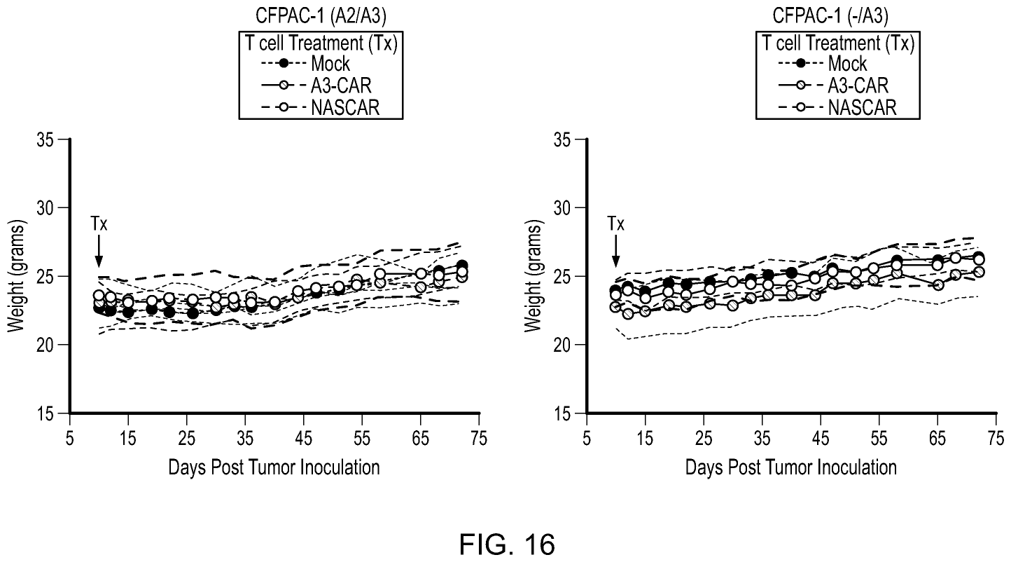

To determine whether the NASCAR approach could be extended to an in vivo setting, constructs were converted from the transient mRNA-based expression system to a stable CRISPR-based knock-in expression system. A knock-in strategy was chosen due to its reported ability to produce tight expression distribution patterns for introduced CAR constructs in primary human T cells. Homology directed repair (HDR) templates comprising an A3-CAR, or a bicistronic NASCAR construct containing an A3-CAR and A2-iCAR with an intervening 2A self-cleaving peptide sequence, were generated. The iCAR was placed 5′ to the CAR to skew the ratio in favor of the antecedent receptor. The HDR template was targeted to the B2M locus to allow for high levels of expression, and a guide RNA (gRNA) targeting the T-cell receptor a constant (TRAC) locus was simultaneously included in the electroporation cocktail to inactivate TRAC and consequently reduce alloreactivity toward the human cancer cells used to establish tumors in the mice. In vitro characterization of this stable NASCAR expression system revealed the near-complete ablation of TCR expression, an approximately 30% editing efficiency of the introduced transgenes, and the expected allelic recognition pattern when co-incubated with CFPAC-1 HLA KO isogenic target cell lines (FIGS. 14A-14C). In vivo characterization employed a subcutaneous xenograft model of NOD.Cg-Prkdc^{scid} Il2rg^{tm1Wjl}/SzJ(NSG) mice with CFPAC-1 A2/A3 or -/A3 tumors. For treatment, CRISPR-engineered CAR or NASCAR T cells were administered via tail vein 10 days following tumor inoculation once tumors were established and palpable (FIG. 15A). While treatments with CAR T cells resulted in regression of both tumors, NASCAR T cells eliminated the -/A3 tumor and spared the A2/A3 heterozygous tumor representing normal tissues (FIG. 15B). The treatments were well-tolerated as evidenced by the absence of significant deviations from normal body weight gains (FIG. 16).

### Example 6—Proof-of-Concept synNotch-NASCAR LOH Targeting

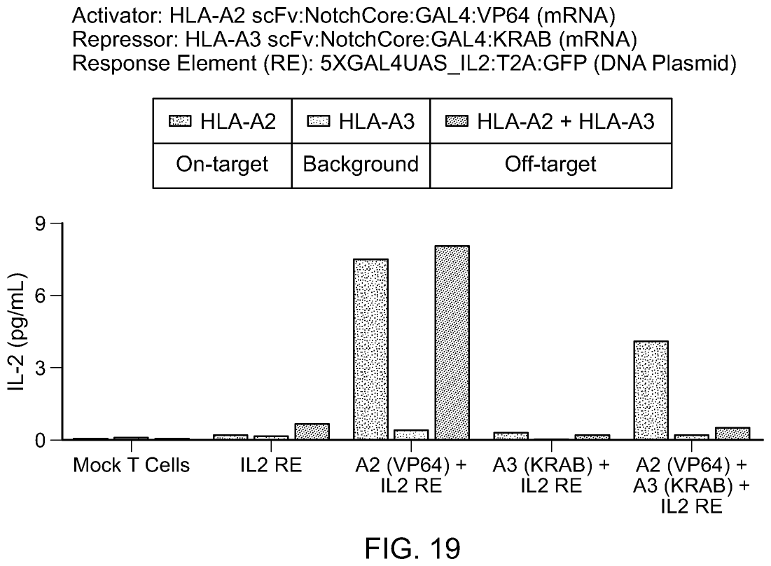

To determine whether synNotch-NASCAR T cells could detect and target LOH, CFPAC-1 cancer cells with the indicated HLA-A allele status were co-incubated with synNotch-NASCAR T cells configured with the indicated allele-targeting synNotch activator and/or repressor mRNA(s) and IL-2 DNA response element combinations. SynNotch-NASCAR activity was assessed by ELISA for IL-2 (FIG. 19).

### Example 7—Flow Characterization of CAR and iCAR Constructs

CAR and iCAR constructs were designed to be introduced into T cells as dsDNA templates via homology directed repair at the B2M locus after a CRISPR Cas9 induced double stranded DNA break. Major components that were used include homology arms, 2A sequences, CAR, iCAR, and polyA terminators (FIG. 21).

TABLE 3

| | Construct | |
|---|---|---|
| | Name | |
| SEQ | [cellular | |
| ID | engineering | |
| NO. | methodology | Amino Acid Sequence |
| 1 | Clone 13 | DIQMTQSPSSLSASVGDRVT |
| | scFv | ITCRASQDVNTAVAWYQQKP |
| | | GKAPKLLIYSASFLYSGVPS |
| | | RFSGSRSGTDFTLTISSLQP |
| | | EDFATYYCQQSYTSPITFGQ |
| | | GTKVEIKRTGGGSGGGGSGG |
| | | GASEVQLVESGGGLVQPGGS |
| | | LRLSCAASGFNLSGTYMHWV |
| | | RQAPGKGLEWVAWFSPYSSY |
| | | TNYADSVKGRFTISADTSKN |
| | | TAYLQMNSLRAEDTAVYYCS |
| | | RGSSHSSVAFDYWGQGTLVT |
| | | VSS |
| 2 | A2-CAR | MALPVTALLLPLALLLHAAR |
| | (Clone BB7.2) | PQVQLQQSGPELVKPGASVK |
| | [mRNA | MSCKASGYTFTSYHIQWVKQ |
| | electroporation] | RPGQGLEWIGWIYPGDGSTQ |
| | | YNEKFKGKTTLTADKSSSTA |
| | | YMLLSSLTSEDSAIYFCARE |
| | | GTYYAMDYWGQGTSVTVSSG |
| | | GGGSGGGGSGGGGSDIVMTQ |
| | | APLSLPVSLGDQVSISCRSS |
| | | QSIVHSNGNTYLEWYLQKPG |
| | | QSPKLLIYKVSNRFSGVPDR |
| | | FSGSGSGTDFTLKISRVEAE |
| | | DLGVYYCFQGSHVPRTFGGG |
| | | TKLELKRTGLSTTTPAPRPP |
| | | TPAPTIASQPLSLRPEACRP |
| | | AAGGAVHTRGLDFACDIYFW |
| | | VLVVVGGVLACYSLLVTVAF |
| | | IIFWVRSKRSRLLHSDYMNM |
| | | TPRRPGPTRKHYQPYAPPRD |
| | | FAAYRSRVKFSRSADAPAYQ |
| | | QGQNQLYNELNLGRREEYDV |
| | | LDKRRGRDPEMGGKPQRRKN |
| | | PQEGLYNELQKDKMAEAYSE |
| | | IGMKGERRRGKGHDGLYQGL |
| | | STATKDTYDALHMQALPPR |
| 3 | A3-CAR | MALPVTALLLPLALLLHAAR |
| | (Clone 13) | PDIQMTQSPSSLSASVGDRV |
| | [mRNA | TITCRASQDVNTAVAWYQQK |
| | electroporation] | PGKAPKLLIYSASFLYSGVP |
| | | SRFSGSRSGTDFTLTISSLQ |
| | | PEDFATYYCQQSYTSPITFG |
| | | QGTKVEIKRTGGGSGGGGSG |
| | | GGASEVQLVESGGGLVQPGG |
| | | SLRLSCAASGENLSGTYMHW |
| | | VRQAPGKGLEWVAWFSPYSS |
| | | YTNYADSVKGRFTISADTSK |
| | | NTAYLQMNSLRAEDTAVYYC |
| | | SRGSSHSSVAFDYWGQGTLV |
| | | TVSSGLSTTTPAPRPPTPAP |
| | | TIASQPLSLRPEACRPAAGG |
| | | AVHTRGLDFACDIYFWVLVV |
| | | VGGVLACYSLLVTVAFIIFW |
| | | VRSKRSRLLHSDYMNMTPRR |
| | | PGPTRKHYQPYAPPRDFAAY |
| | | RSRVKFSRSADAPAYQQGQN |
| | | QLYNELNLGRREEYDVLDKR |
| | | RGRDPEMGGKPQRRKNPQEG |
| | | LYNELQKDKMAEAYSEIGMK |
| | | GERRRGKGHDGLYQGLSTAT |
| | | KDTYDALHMQALPPR |
| 4 | A2-iCAR | MALPVTALLLPLALLLHAAR |
| | (Clone BB7.2) | PQVQLQQSGPELVKPGASVK |
| | [mRNA | MSCKASGYTFTSYHIQWVKQ |
| | electroporation] | RPGQGLEWIGWIYPGDGSTQ |
| | | YNEKFKGKTTLTADKSSSTA |
| | | YMLLSSLTSEDSAIYFCARE |
| | | GTYYAMDYWGQGTSVTVSSG |
| | | GGGSGGGGSGGGGSDIVMTQ |
| | | APLSLPVSLGDQVSISCRSS |
| | | QSIVHSNGNTYLEWYLQKPG |
| | | QSPKLLIYKVSNRFSG5VPD |
| | | RFSGSGSGTDFTLKISRVEA |
| | | EDLGVYYCFQGSHV6PRTFG |
| | | GGTKLELKRTGLSTTTPAPR |
| | | PPTPAPTIASQPL7SLRPEA |
| | | CRPAAGGAVHTRGLDFACDI |
| | | YIWAPLAGTC8GVLLLSLVI |
| | | TLYCNHRCSRA |
| | | ARGTIGARRTGQPLKEDPSA |
| | | VPVFSVDYGELDFQWREKTP |
| | | EPPVPCVPEQTEYATIVFPS |
| | | GMGTSSPARRGSADGPRSAQ |
| | | PLRPEDGHCSWPL |
| 5 | A3-iCAR | MALPVTALLLPLALLLHAAR |
| | (Clone 13) | PDIQMTQSPSSLSASVGDRV |
| | [mRNA | TITCRASQDVNTAVAWYQQK |
| | electroporation] | PGKAPKLLIYSASFLYSGVP |
| | | SRFSGSRSGTDFTLTISSLQ |
| | | PEDFATYYCQQSYTSPITFG |
| | | QGTKVEIKRTGGGSGGGGSG |
| | | GGASEVQLVESGGGLVQPGG |
| | | SLRLSCAASGENLSGTYMHW |
| | | VRQAPGKGLEWVAWFSPYSS |
| | | YTNYADSVKGRFTISADTSK |
| | | NTAYLQMNSLRAEDTAVYYC |
| | | SRGSSHSSVAFDYWGQGTLV |
| | | TVSSGLSTTTPAPRPPTPAP |
| | | TIASQPLSLRPEACRPAAGG |
| | | AVHTRGLDFACDIYIWAPLA |
| | | GTCGVLLLSLVITLYCNHRC |
| | | SRAARGTIGARRTGQPLKED |
| | | PSAVPVFSVDYGELDFQWRE |
| | | KTPEPPVPCVPEQTEYATIV |
| | | FPSGMGTSSPARRGSADGPR |
| | | SAQPLRPEDGHCSWPL |
| 6 | B2M--A2-iCAR- | RAKRSGSGATNFSLLKQAGD |
| | 2A-A3-CAR | VEENPGPMALPVTALLLPLA |
| | [CRISPR HDR | LLLHAARPQVQLQQSGPELV |
| | knock-in] | KPGASVKMSCKASGYTFTSY |
| | | HIQWVKQRPGQGLEWIGWIY |
| | | PGDGSTQYNEKFKGKTTLTA |
| | | DKSSSTAYMLLSSLTSEDSA |
| | | IYFCAREGTYYAMDYWGQGT |
| | | SVTVSSGGGGSGGGGSGGGG |
| | | SDIVMTQAPLSLPVSLGDQV |
| | | SISCRSSQSIVHSNGNTYLE |
| | | WYLQKPGQSPKLLIYKVSNR |
| | | FSGVPDRFSGSGSGTDFTLK |
| | | ISRVEAEDLGVYYCFQGSHV |
| | | PRTFGGGTKLELKRTGLSTT |
| | | TPAPRPPTPAPTIASQPLSL |
| | | RPEACRPAAGGAVHTRGLDF |
| | | ACDIYIWAPLAGTCGVLLLS |
| | | LVITLYCNHRCSRAARGTIG |
| | | ARRTGQPLKEDPSAVPVFSV |
| | | DYGELDFQWREKTPEPPVPC |
| | | VPEQTEYATIVFPSGMGTSS |
| | | PARRGSADGPRSAQPLRPED |
| | | GHCSWPLRAKRSGSGATNFS |
| | | LLKQAGDVEENPGPMALPVT |
| | | ALLLPLALLLHAARPDIQMT |
| | | QSPSSLSASVGDRVTITCRA |
| | | SQDVNTAVAWYQQKPGKAPK |
| | | LLIYSASFLYSGVPSRFSGS |
| | | RSGTDFTLTISSLQPEDFAT |
| | | YYCQQSYTSPITFGQGTKVE |
| | | IKRTGGGSGGGGSGGGASEV |
| | | QLVESGGGLVQPGGSLRLSC |
| | | AASGFNLSGTYMHWVRQAPG |
| | | KGLEWVAWFSPYSSYTNYAD |
| | | SVKGRFTISADTSKNTAYLQ |
| | | MNSLRAEDTAVYYCSRGSSH |
| | | SSVAFDYWGQGTLVTVSSGL |
| | | STTTPAPRPPTPAPTIASQP |
| | | LSLRPEACRPAAGGAVHTRG |
| | | LDFACDIYFWVLVVVGGVLA |
| | | CYSLLVTVAFIIFWVRSKRS |
| | | RLLHSDYMNMTPRRPGPTRK |
| | | HYQPYAPPRDFAAYRSRVKF |
| | | SRSADAPAYQQGQNQLYNEL |
| | | NLGRREEYDVLDKRRGRDPE |
| | | MGGKPQRRKNPQEGLYNELQ |
| | | KDKMAEAYSEIGMKGERRRG |
| | | KGHDGLYQGLSTATKDTYDA |
| | | LHMQALPPR |
| 7 | B2M--A3-CAR | RAKRSGSGATNFSLLKQAGD |
| | [CRISPR HDR | VEENPGPMALPVTALLLPLA |
| | knock-in] | LLLHAARPDIQMTQSPSSLS |
| | | ASVGDRVTITCRASQDVNTA |
| | | VAWYQQKPGKAPKLLIYSAS |
| | | FLYSGVPSRFSGSRSGTDFT |
| | | LTISSLQPEDFATYYCQQSY |
| | | TSPITFGQGTKVEIKRTGGG |
| | | SGGGGSGGGASEVQLVESGG |
| | | GLVQPGGSLRLSCAASGFNL |
| | | SGTYMHWVRQAPGKGLEWVA |
| | | WFSPYSSYTNYADSVKGRFT |
| | | ISADTSKNTAYLQMNSLRAE |
| | | DTAVYYCSRGSSHSSVAFDY |
| | | WGQGTLVTVSSGLSTTTPAP |
| | | RPPTPAPTIASQPLSLRPEA |
| | | CRPAAGGAVHTRGLDFACDI |
| | | YFWVLVVVGGVLACYSLLVT |
| | | VAFIIFWVRSKRSRLLHSDY |
| | | MNMTPRRPGPTRKHYQPYAP |
| | | PRDFAAYRSRVKFSRSADAP |
| | | AYQQGQNQLYNELNLGRREE |
| | | YDVLDKRRGRDPEMGGKPQR |
| | | RKNPQEGLYNELQKDKMAEA |
| | | YSEIGMKGERRRGKGHDGLY |
| | | QGLSTATKDTYDALHMQALP |
| | | PR |
| 8 | CD28H/TM-A2- | RAKRSGSGATNFSLLKQAGD |
| | ICdel-2A- | VEENPGPMALPVTALLLPLA |
| | A3-CAR | LLLHAARPQVQLQQSGPELV |
| | [CRISPR HDR | KPGASVKMSCKASGYTFTSY |
| | knock-in] | HIQWVKQRPGQGLEWIGWIY |
| | | PGDGSTQYNEKFKGKTTLTA |
| | | DKSSSTAYMLLSSLTSEDSA |
| | | IYFCAREGTYYAMDYWGQGT |
| | | SVTVSSGGGGSGGGGSGGGG |
| | | SDIVMTQAPLSLPVSLGDQV |
| | | SISCRSSQSIVHSNGNTYLE |
| | | WYLQKPGQSPKLLIYKVSNR |
| | | FSGVPDRFSGSGSGTDFTLK |
| | | ISRVEAEDLGVYYCFQGSHV |
| | | PRTFGGGTKLELKRAAAPTT |
| | | TPAPRPPTPAPTIASQPLSL |
| | | RPEACRPAAGGAVHTRGLDF |
| | | ACDIYIWAPLAGTCGVLLLS |
| | | LVITLYCNHRMHRAKRSGSG |
| | | ATNFSLLKQAGDVEENPGPM |
| | | ALPVTALLLPLALLLHAARP |
| | | DIQMTQSPSSLSASVGDRVT |
| | | ITCRASQDVNTAVAWYQQKP |
| | | GKAPKLLIYSASFLYSGVPS |
| | | RFSGSRSGTDFTLTISSLQP |
| | | EDFATYYCQQSYTSPITFGQ |
| | | GTKVEIKRTGGGSGGGGSGG |
| | | GASEVQLVESGGGLVQPGGS |
| | | LRLSCAASGFNLSGTYMHWV |
| | | RQAPGKGLEWVAWFSPYSSY |
| | | TNYADSVKGRFTISADTSKN |
| | | TAYLQMNSLRAEDTAVYYCS |
| | | RGSSHSSVAFDYWGQGTLVT |
| | | VSSAAAIEVMYPPPYLDNEK |
| | | SNGTIIHVKGKHLCPSPLFP |
| | | GPSKPFWVLVVVGGVLACYS |
| | | LLVTVAFIIFWVRSKRSRLL |
| | | HSDYMNMTPRRPGPTRKHYQ |
| | | PYAPPRDFAAYRSRVKFSRS |
| | | ADAPAYQQGQNQLYNELNLG |
| | | RREEYDVLDKRRGRDPEMGG |
| | | KPRRKNPQEGLYNELQKDKM |
| | | AEAYSEIGMKGERRRGKGHD |
| | | GLYQGLSTATKDTYDALHMQ |
| | | ALPPR |
| 9 | CD28H/TM-A2- | RAKRSGSGATNFSLLKQAGD |
| | iCAR-2A- | VEENPGPMALPVTALLLPLA |
| | A3-CAR | LLLHAARPQVQLQQSGPELV |
| | [CRISPR HDR | KPGASVKMSCKASGYTFTSY |
| | knock-in] | HIQWVKQRPGQGLEWIGWIY |
| | | PGDGSTQYNEKFKGKTTLTA |
| | | DKSSSTAYMLLSSLTSEDSA |
| | | IYFCAREGTYYAMDYWGQGT |
| | | SVTVSSGGGGSGGGGSGGGG |
| | | SDIVMTQAPLSLPVSLGDQV |
| | | SISCRSSQSIVHSNGNTYLE |
| | | WYLQKPGQSPKLLIYKVSNR |
| | | FSGVPDRFSGSGSGTDFTLK |
| | | ISRVEAEDLGVYYCFQGSHV |
| | | PRTFGGGTKLELKRAAAPTT |
| | | TPAPRPPTPAPTIASQPLSL |
| | | RPEACRPAAGGAVHTRGLDF |
| | | ACDIYIWAPLAGTCGVLLLS |
| | | LVITLYCNHRMHCSRAARGT |
| | | IGARRTGQPLKEDPSAVPVF |
| | | SVDYGELDFQWREKTPEPPV |
| | | PCVPEQTEYATIVFPSGMGT |
| | | SSPARRGSADGPRSAQPLRP |
| | | EDGHCSWPLRAKRSGSGATN |
| | | FSLLKQAGDVEENPGPMALP |
| | | VTALLLPLALLLHAARPDIQ |
| | | MTQSPSSLSASVGDRVTITC |
| | | RASQDVNTAVAWYQQKPGKA |
| | | PKLLIYSASFLYSGVPSRFS |
| | | GSRSGTDFTLTISSLQPEDF |
| | | ATYYCQQSYTSPITFGQGTK |
| | | VEIKRTGGGSGGGGSGGGAS |
| | | EVQLVESGGGLVQPGGSLRL |
| | | SCAASGFNLSGTYMHWVRQA |
| | | PGKGLEWVAWFSPYSSYTNY |
| | | ADSVKGRFTISADTSKNTAY |
| | | LQMNSLRAEDTAVYYCSRGS |
| | | SHSSVAFDYWGQGTLVTVSS |
| | | AAAIEVMYPPPYLDNEKSNG |
| | | TIIHVKGKHLCPSPLFPGPS |
| | | KPFWVLVVVGGVLACYSLLV |
| | | TVAFIIFWVRSKRSRLLHSD |
| | | YMNMTPRRPGPTRKHYQPYA |
| | | PPRDFAAYRSRVKFSRSADA |
| | | PAYQQGQNQLYNELNLGRRE |
| | | EYDVLDKRRGRDPEMGGKPR |
| | | RKNPQEGLYNELQKDKMAEA |
| | | YSEIGMKGERRRGKGHDGLY |
| | | QGLSTATKDTYDALHMQALP |
| | | PR |
| 10 | CD28H/TM- | RAKRSGSGATNFSLLKQAGD |
| | 2xA2-iCAR- | VEENPGPMALPVTALLLPLA |
| | 2A-A3-CAR | LLLHAARPQVQLQQSGPELV |
| | [CRISPR HDR | KPGASVKMSCKASGYTFTSY |
| | knock-in] | HIQWVKQRPGQGLEWIGWIY |
| | | PGDGSTQYNEKFKGKTTLTA |
| | | DKSSSTAYMLLSSLTSEDSA |
| | | IYFCAREGTYYAMDYWGQGT |
| | | SVTVSSGGGGSGGGGSGGGG |
| | | SDIVMTQAPLSLPVSLGDQV |
| | | SISCRSSQSIVHSNGNTYLE |
| | | WYLQKPGQSPKLLIYKVSNR |
| | | FSGVPDRFSGSGSGTDFTLK |
| | | ISRVEAEDLGVYYCFQGSHV |
| | | PRTFGGGTKLELKRAAAPTT |
| | | TPAPRPPTPAPTIASQPLSL |
| | | RPEACRPAAGGAVHTRGLDF |
| | | ACDIYIWAPLAGTCGVLLLS |
| | | LVITLYCNHRMHCSRAARGT |
| | | IGARRTGQPLKEDPSAVPVF |
| | | SVDYGELDFQWREKTPEPPV |
| | | PCVPEQTEYATIVFPSGMGT |
| | | SSPARRGSADGPRSAQPLRP |
| | | EDGHCSWPLRAKRSGSGQCT |
| | | NYALLKLAGDVESNPGPMAL |
| | | PVTALLLPLALLLHAARPQV |
| | | QLQQSGPELVKPGASVKMSC |
| | | KASGYTFTSYHIQWVKQRPG |
| | | QGLEWIGWIYPGDGSTQYNE |
| | | KFKGKTTLTADKSSSTAYML |
| | | LSSLTSEDSAIYFCAREGTY |
| | | YAMDYWGQGTSVTVSSGGGG |
| | | SGGGGSGGGG |
| | | SDIVMTQAPLSLPVSLGDQV |
| | | SISCRSSQSIVHSNGNTYLE |
| | | WYLQKPGQSPKLLIYKVSNR |
| | | FSGVPDRFSGSGSGTDFTLK |
| | | ISRVEAEDLGVYYCFQGSHV |
| | | PRTFGGGTKLELKRAAAPTT |
| | | TPAPRPPTPAPTIASQPLSL |
| | | RPEACRPAAGGAVHTRGLDF |
| | | ACDIYIWAPLAGTCGVLLLS |
| | | LVITLYCNHRMHCSRAARGT |
| | | IGARRTGQPLKEDPSAVPVF |
| | | SVDYGELDFQWREKTPEPPV |
| | | PCVPEQTEYATIVFPSGMGT |
| | | SSPARRGSADGPRSAQPLRP |
| | | EDGHCSWPLRAKRSGSGATN |
| | | FSLLKQAGDVEENPGPMALP |
| | | VTALLLPLALLLHAARPDIQ |
| | | MTQSPSSLSASVGDRVTITC |
| | | RASQDVNTAVAWYQQKPGKA |
| | | PKLLIYSASFLYSGVPSRFS |
| | | GSRSGTDFTLTISSLQPEDF |
| | | ATYYCQQSYTSPITFGQGTK |
| | | VEIKRTGGGSGGGGSGGGAS |
| | | EVQLVESGGGLVQPGGSLRL |
| | | SCAASGFNLSGTYMHWVRQA |
| | | PGKGLEWVAWFSPYSSYTNY |
| | | ADSVKGRFTISADTSKNTAY |
| | | LQMNSLRAEDTAVYYCSRGS |
| | | SHSSVAFDYWGQGTLVTVSS |
| | | AAAIEVMYPPPYLDNEKSNG |
| | | TIIHVKGKHLCPSPLFPGPS |
| | | KPFWVLVVVGGVLACYSLLV |
| | | TVAFIIFWVRSKRSRLLHSD |
| | | YMNMTPRRPGPTRKHYQPYA |
| | | PPRDFAAYRSRVKFSRSADA |
| | | PAYQQGQNQLYNELNLGRRE |
| | | EYDVLDKRRGRDPEMGGKPR |
| | | RKNPQEGLYNELQKDKMAEA |
| | | YSEIGMKGERRRGKGHDGLY |
| | | QGLSTATKDTYDALHMQALP |
| | | PR |
| 11 | CD8H/TM-A3- | RAKRSGSGATNFSLLKQAGD |
| | CAR-2A- | VEENPGPMALPVTALLLPLA |
| | A2-ICdel | LLLHAARPDIQMTQSPSSLS |
| | [CRISPR HDR | ASVGDRVTITCRASQDVNTA |
| | knock-in] | VAWYQQKPGKAPKLLIYSAS |
| | | FLYSGVPSRFSGSRSGTDFT |
| | | LTISSLQPEDFATYYCQQSY |
| | | TSPITFGQGTKVEIKRTGGG |
| | | SGGGGSGGGASEVQLVESGG |
| | | GLVQPGGSLRLSCAASGFNL |
| | | SGTYMHWVRQAPGKGLEWVA |
| | | WFSPYSSYTNYADSVKGRFT |
| | | ISADTSKNTAYLQMNSLRAE |
| | | DTAVYYCSRGSSHSSVAFDY |
| | | WGQGTLVTVSSDPTTTPAPR |
| | | PPTPAPTIASQPLSLRPEAC |
| | | RPAAGGAVHTRGLDFACDIF |
| | | WVLVVVGGVLACYSLLVTVA |
| | | FIIFWVRSKRSRLLHSDYMN |
| | | MTPRRPGPTRKHYQPYAPPR |
| | | DFAAYRSRVKFSRSADAPAY |
| | | QQGQNQLYNELNLGRREEYD |
| | | VLDKRRGRDPEMGGKPRRKN |
| | | PQEGLYNELQKDKMAEAYSE |
| | | IGMKGERRRGKGHDGLYQGL |
| | | STATKDTYDALHMQALPPRR |
| | | AKRSGSGATNFSLLKQAGDV |
| | | EENPGPMALPVTALLLPLAL |
| | | LLHAARPQVQLQQSGPELVK |
| | | PGASVKMSCKASGYTFTSYH |
| | | IQWVKQRPGQGLEWIGWIYP |
| | | GDGSTQYNEKFKGKTTLTAD |
| | | KSSSTAYMLLSSLTSEDSAI |
| | | YFCAREGTYYAMDYWGQGTS |
| | | VTVSSGGGGSGGGGGGGGSD |
| | | IVMTQAPLSLPVSLGDQVSI |
| | | SCRSSQSIVHSNGNTYLEWY |
| | | LQKPGQSPKLLIYKVSNRFS |
| | | GVPDRFSGSGSGTDFTLKIS |
| | | RVEAEDLGVYYCFQGSHVPR |
| | | TFGGGTKLELKMDPATTTKP |
| | | VLRTPSPVHPTGTSQPQRPE |
| | | DCRPRGSVKGTGLDFACDIY |
| | | IWAPLAGICVALLLSLIITL |
| | | ICYHRSRKRVCK |
| 12 | CD8H/TM-A3- | RAKRSGSGATNFSLLKQAGD |
| | CAR-2A- | VEENPGPMALPVTALLLPLA |
| | A2-SHP1 | LLLHAARPDIQMTQSPSSLS |
| | [CRISPR HDR | ASVGDRVTITCRASQDVNTA |
| | knock-in] | VAWYQQKPGKAPKLLIYSAS |
| | | FLYSGVPSRFSGSRSGTDFT |
| | | LTISSLQPEDFATYYCQQSY |
| | | TSPITFGQGTKVEIKRTGGG |
| | | SGGGGSGGGASEVQLVESGG |
| | | GLVQPGGSLRLSCAASGENL |
| | | SGTYMHWVRQAPGKGLEWVA |
| | | WFSPYSSYTNYADSVKGRFT |
| | | ISADTSKNTAYLQMNSLRAE |
| | | DTAVYYCSRGSSHSSVAFDY |
| | | WGQGTLVTVSSDPTTTPAPR |
| | | PPTPAPTIASQPLSLRPEAC |
| | | RPAAGGAVHTRGLDFACDIF |
| | | WVLVVVGGVLACYSLLVTVA |
| | | FIIFWVRSKRSRLLHSDYMN |
| | | MTPRRPGPTRKHYQPYAPPR |
| | | DFAAYRSRVKFSRSADAPAY |
| | | QQGQNQLYNELNLGRREEYD |
| | | VLDKRRGRDPEMGGKPRRKN |
| | | PQEGLYNELQKDKMAEAYSE |
| | | IGMKGERRRGKGHDGLYQGL |
| | | STATKDTYDALHMQALPPRR |
| | | AKRSGSGATNFSLLKQAGDV |
| | | EENPGPMALPVTALLLPLAL |
| | | LLHAARPQVQLQQSGPELVK |
| | | PGASVKMSCKASGYTFTSYH |
| | | IQWVKQRPGQGLEWIGWIYP |
| | | GDGSTQYNEKFKGKTTLTAD |
| | | KSSSTAYMLLSSLTSEDSAI |
| | | YFCAREGTYYAMDYWGQGTS |
| | | VTVSSGGGGSGGGGSGGGGS |
| | | DIVMTQAPLSLPVSLGDQVS |
| | | ISCRSSQSIVHSNGNTYLEW |
| | | YLQKPGQSPKLLIYKVSNRF |
| | | SGVPDRFSGSGSGTDFTLKI |
| | | SRVEAEDLGVYYCFQGSHVP |
| | | RTFGGGTKLELKMDPATTTK |
| | | PVLRTPSPVHPTGTSQPQRP |
| | | EDCRPRGSVKGTGLDFACDI |
| | | YIWAPLAGICVALLLSLIIT |
| | | LICYHRSRKRVCKSGGGSFW |
| | | EEFESLQKQEVKNLHQRLEG |
| | | QRPENKGKNRYKNILPFDHS |
| | | RVILQGRDSNIPGSDYINAN |
| | | YIKNQLLGPDENAKTYIASQ |
| | | GCLEATVNDFWQMAWQENSR |
| | | VIVMTTREVEKGRNKCVPYW |
| | | PEVGMQRAYGPYSVTNCGEH |
| | | DTTEYKLRTLQVSPLDNGDL |
| | | IREIWHYQYLSWPDHGVPSE |
| | | PGGVLSFLDQINQRQESLPH |
| | | AGPIIVHCSAGIGRTGTIIV |
| | | IDMLMENISTKGLDCDIDIQ |
| | | KTIQMVRAQRSGMVQTEAQY |
| | | KFIYVAIAQFIETTKKKL |
| 13 | CD8H/TM-A3- | RAKRSGSGATNFSLLKQAGD |
| | CAR-2A- | VEENPGPMALPVTALLLPLA |
| | A2-CD148 | LLLHAARPDIQMTQSPSSLS |
| | [CRISPR HDR | ASVGDRVTITCRASQDVNTA |
| | knock-in] | VAWYQQKPGKAPKLLIYSAS |
| | | FLYSGVPSRFSGSRSGTDFT |
| | | LTISSLQPEDFATYYCQQSY |
| | | TSPITFGQGTKVEIKRTGGG |
| | | SGGGGSGGGASEVQLVESGG |
| | | GLVQPGGSLRLSCAASGFNL |
| | | SGTYMHWVRQAPGKGLEWVA |
| | | WFSPYSSYTNYADSVKGRFT |
| | | ISADTSKNTAYLQMNSLRAE |
| | | DTAVYYCSRGSSHSSVAFDY |
| | | WGQGTLVTVSSDPTTTPAPR |
| | | PPTPAPTIASQPLSLRPEAC |
| | | RPAAGGAVHTRGLDFACDIF |
| | | WVLVVVGGVLACYSLLVTVA |
| | | FIIFWVRSKRSRLLHSDYMN |
| | | MTPRRPGPTRKHYQPYAPPR |
| | | DFAAYRSRVKFSRSADAPAY |
| | | QQGQNQLYNELNLGRREEYD |
| | | VLDKRRGRDPEMGGKPRRKN |
| | | PQEGLYNELQKDKMAEAYSE |
| | | IGMKGERRRGKGHDGLYQGL |
| | | STATKDTYDALHMQALPPRR |
| | | AKRSGSGATNFSLLKQAGDV |
| | | EENPGPMALPVTALLLPLAL |
| | | LLHAARPQVQLQQSGPELVK |
| | | PGASVKMSCKASGYTFTSYH |
| | | IQWVKQRPGQGLEWIGWIYP |
| | | GDGSTQYNEKFKGKTTLTAD |
| | | KSSSTAYMLLSSLTSEDSAI |
| | | YFCAREGTYYAMDYWGQGTS |
| | | VTVSSGGGGSGGGGSGGGGS |
| | | DIVMTQAPLSLPVSLGDQVS |
| | | ISCRSSQSIVHSNGNTYLEW |
| | | YLQKPGQSPKLLIYKVSNRF |
| | | SGVPDRFSGSGSGTDFTLKI |
| | | SRVEAEDLGVYYCFQGSHVP |
| | | RTFGGGTKLELKMDPATTTK |
| | | PVLRTPSPVHPTGTSQPQRP |
| | | EDCRPRGSVKGTGLDFACDI |
| | | LIGVSVVFLFCLLLLVLFCL |
| | | HRQNQIKQGPPRSKDEEQKP |
| | | QQRPDLAVDVLERTADKATV |
| | | NGLPEKDRETDTSALAAGSS |
| | | QEVTYAQLDHWALTQRTARA |
| | | VSPQSTKPMAESITYAAVAR |
| | | HRAEGRGSLLTCGDVEENPG |
| | | PWYHGHMSGGQAETLLQAKG |
| | | EPWTFLVRESLSQPGDFVLS |
| | | VLSDQPKAGPGSPLRVTHIK |
| | | VMCEGGRYTVGGLETFDSLT |
| | | DLVEHFKKTGIEEASGAFVY |
| | | LRQPYSGGGGSFEAYFKKQQ |
| | | ADSNCGFAEEYEDLKLVGIS |
| | | QPKYAAELAENRGKNRYNNV |
| | | LPYDISRVKLSVQTHSTDDY |
| | | INANYMPGYHSKKDFIATQG |
| | | PLPNTLKDFWRMVWEKNVYA |
| | | IIMLTKCVEQGRTKCEEYWP |
| | | SKQAQDYGDITVAMTSEIVL |
| | | PEWTIRDFTVKNIQTSESHP |
| | | LRQFHFTSWPDHGVPDTTDL |
| | | LINFRYLVRDYMKQSPPESP |
| | | ILVHCSAGVGRTGTFIAIDR |
| | | LIYQIENENTVDVYGIVYDL |
| | | RMHRPLMVQTEDQYVFLNQC |
| | | VLDIVRSQKDSKVDLIYQNT |
| | | TAMTIYENLAPVTTFGKTNG |
| | | YIASGS |

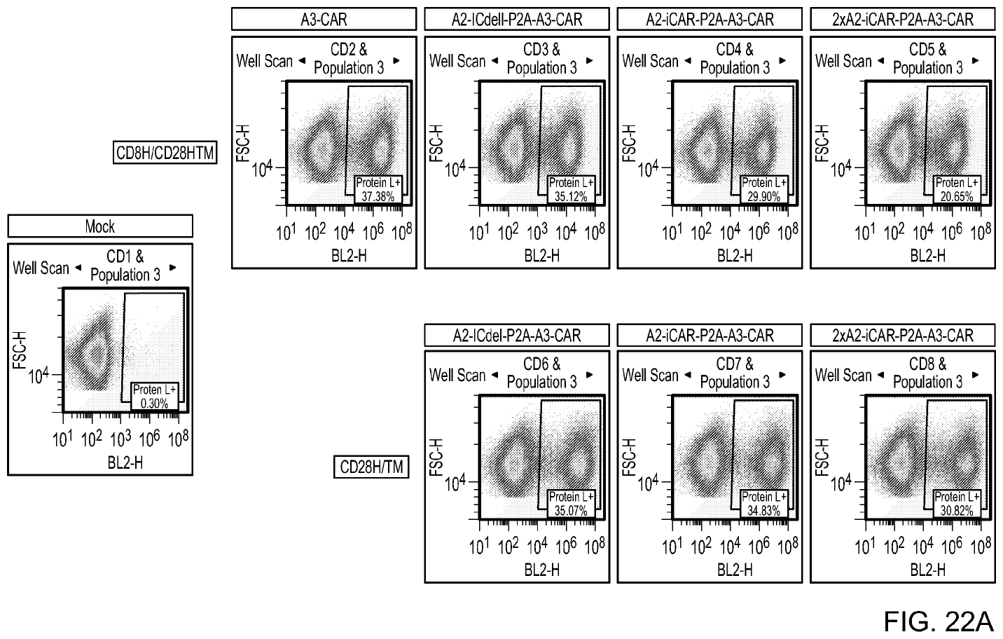

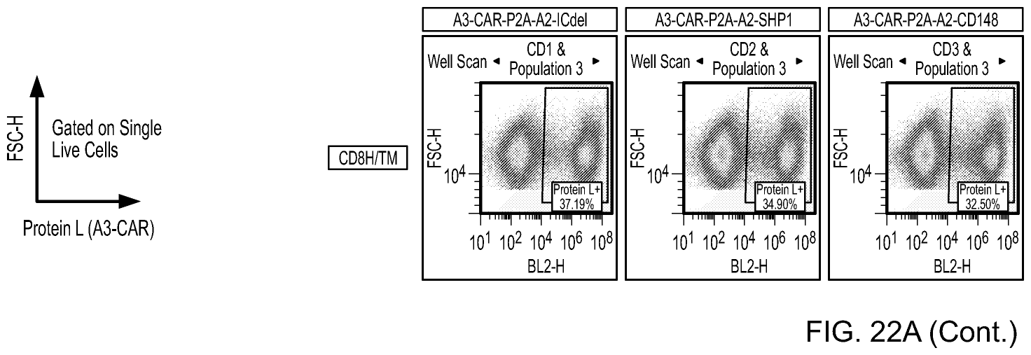

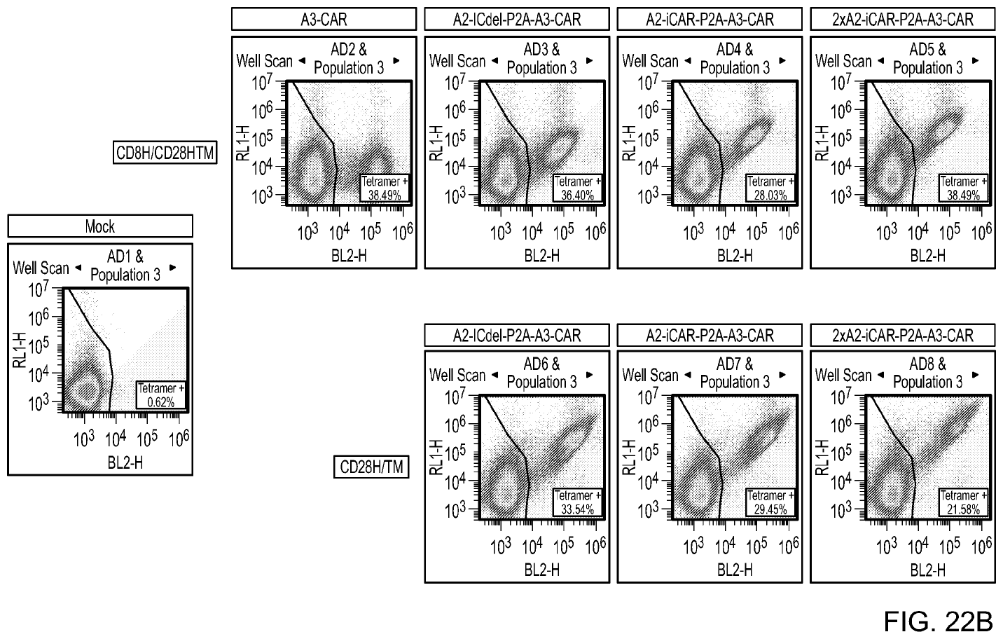

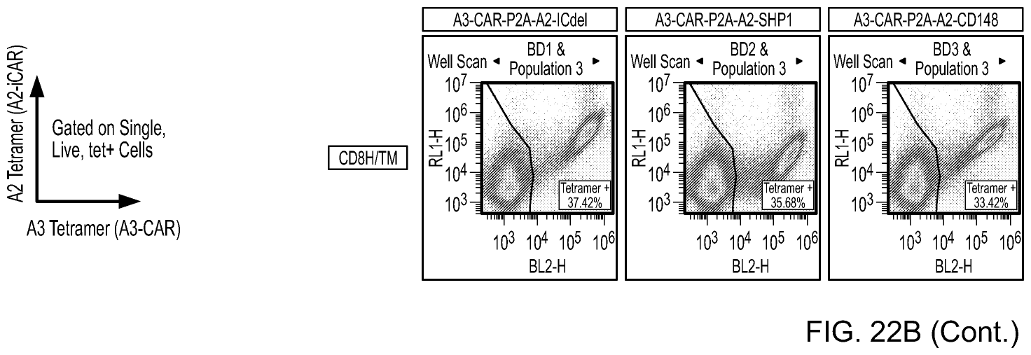

Then, the CAR and iCAR constructs were introduced into primary human T cells via electroporation of a Cas9 RNP targeted to the B2M locus and dsDNA homology directed repair templates. T cells were assayed via flow cytometry 6 days after initial electroporation, wherein Protein L labeling of single live cells (labels A3-CAR) (FIG. 22A), and A2 and A3 tetramer staining of single, live cells was measured (FIG. 22B).

### Example 8—Co-Culture Assessment of CAR and iCAR Combinations

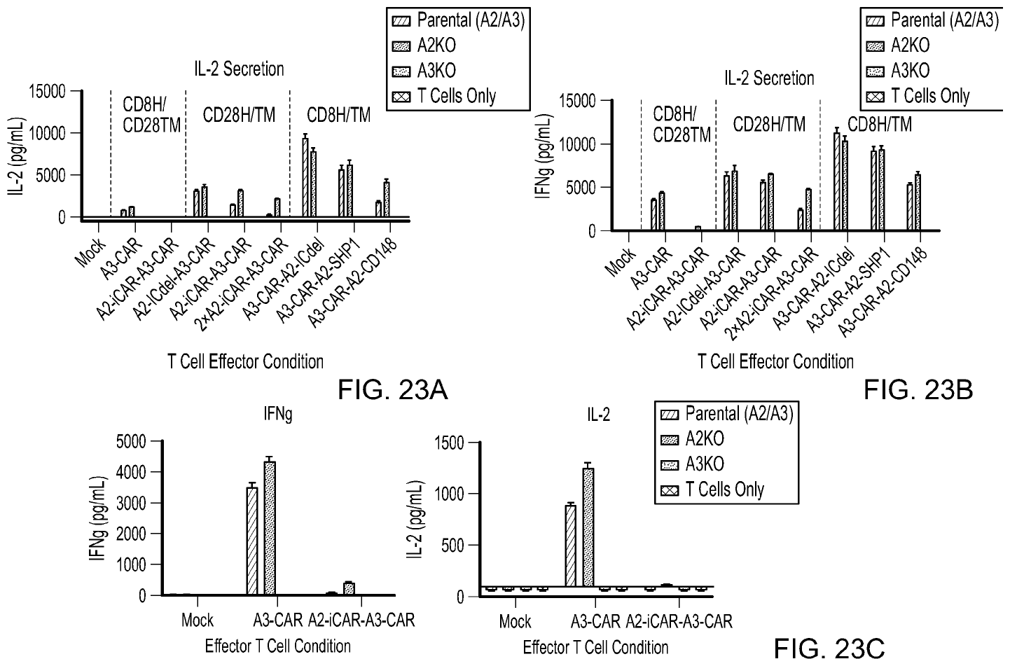

CAR and iCAR modified T cells were assessed in co-culture with three different cell lines to determine iCAR mediated inhibitory potency: CFPAC-1 parental line (HLA-A2+/HLA-A3+), CFPAC-1 A2KO (HLA-A2-/HLA-A3+), and CFPAC-1 A3KO (HLA-A2+/HLA-A3−). Prior to the co-culture, T cells were washed in cytokine free media and then incubated for 16 hours without cytokines. In each well, 60 k CAR/iCAR modified T cells were incubated with 30 k target cells without cytokines for 20 hours. IL-2 (FIG. 23A) and IFNg (FIG. 23B) in cell culture supernatants were quantified via ELISA. FIG. 23C displays IL-2 and IFNg concentrations secreted by only the CD8hinge/transmembrane constructs.

### Example 9—Strategies to Modify CAR:iCAR Expression Ratio

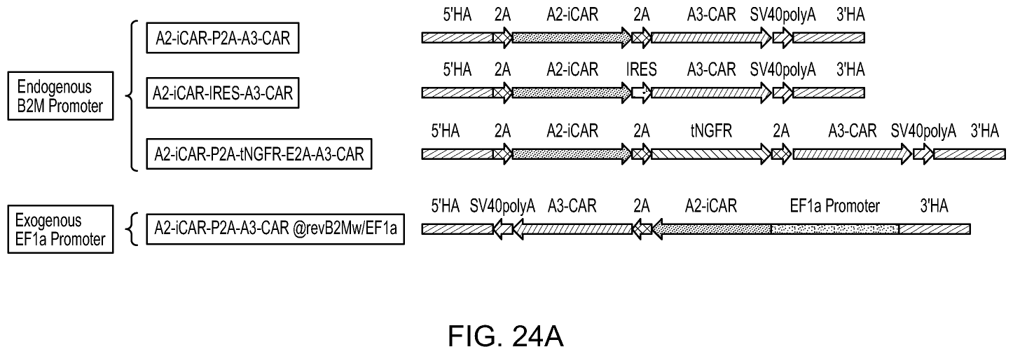

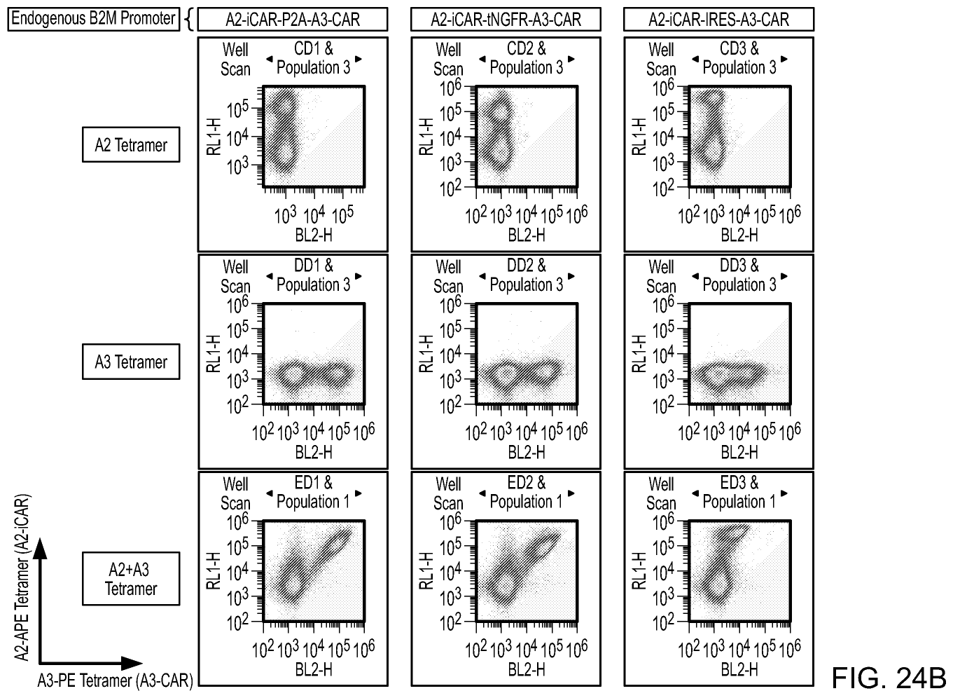

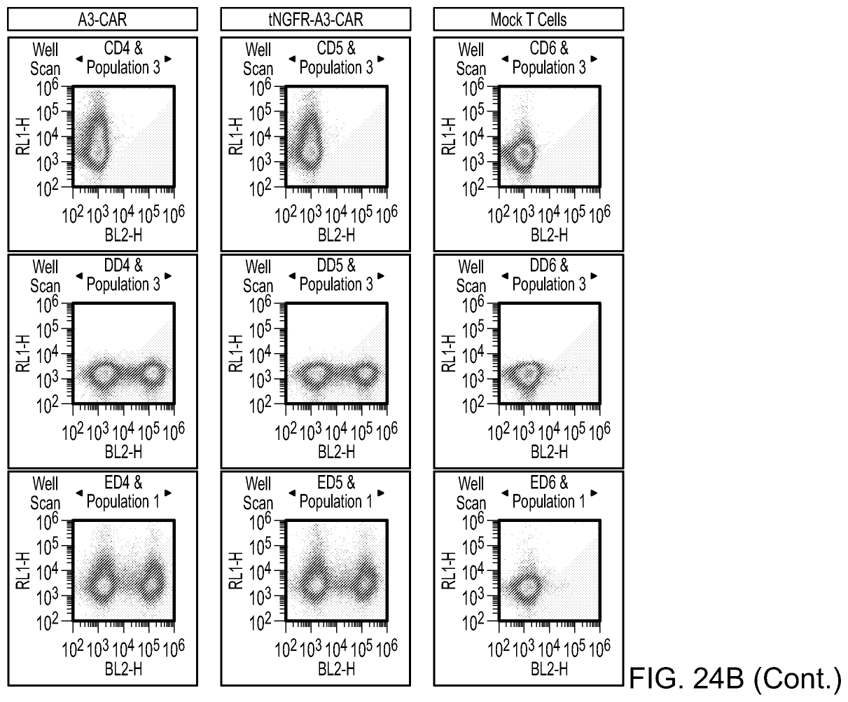

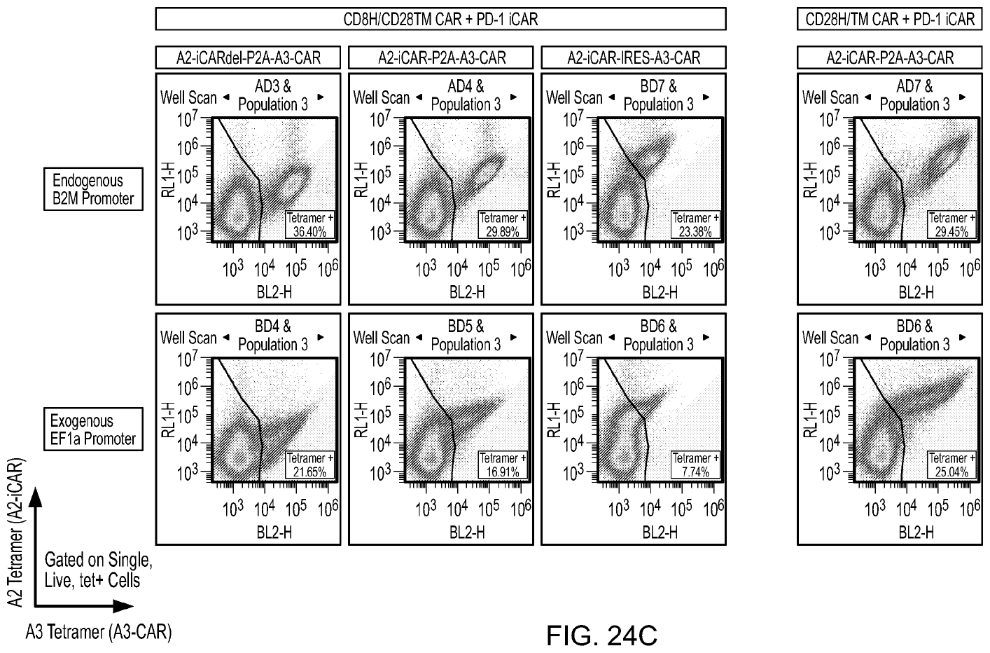

Homology directed repair templates (HDRTs) were designed to skew CAR:iCAR expression ratios (FIG. 24A). The constructs were introduced into primary human T cells via electroporation of a Cas9 RNP targeted to the B2M locus and dsDNA HDRT encoding the specified constructs. Modified T cells were stained with an A3 tetramer to detect CAR expression and an A2 tetramer to detect iCAR expression. All constructs were expressed from an endogenous B2M promoter in (FIG. 24B), whereas comparisons between an endogenous B2M promoter and an exogenous EF1a promoter are shown in (FIG. 24C).

What is claimed is:

1. An immune cell comprising:
(a) a chimeric antigen receptor (CAR), wherein the CAR comprises an extracellular antigen binding domain that binds specifically to a first epitope; and
(b) an inhibitory chimeric antigen receptor (iCAR), wherein the iCAR comprises an extracellular antigen binding domain that binds specifically to a second epitope, wherein the immune cell is activated when the immune cells binds to the first epitope and does not bind to the second epitope; and
wherein the immune cell is inactivated when the immune cell binds to the first and second epitopes.

2. The immune cell of claim 1, wherein the immune cell is a T cell.

3. The immune cell of claim 1 or 2, wherein the first epitope is expressed from a first allele and the second epitope is expressed from a second allele, wherein the first allele and the second allele are from a same gene.

4. The immune cell of claim 3, wherein the first epitope, the second epitope, or both is expressed from a human leukocyte antigen (HLA) gene.

5. The immune cell of any one of claims 1-4, wherein the first epitope is a tumor-associated antigen (TAA).

6. The immune cell of any one of claims 1-5, wherein the second epitope is expressed from a polymorphic allele that is lost in a cancer cell but present in a normal cell.

7. The immune cell of any one of claims 1-6, wherein the first epitope, the second epitope, or both is present in an extracellular domain of a cell surface protein.

8. The immune cell of any one of claims 1-6, wherein the first epitope, the second epitope, or both is present in an intracellular protein that is presented on the surface of a cell by an HLA molecule.

9. The immune cell of any one of claims 1-8, wherein a transmembrane domain of the CAR comprises a CD8-alpha transmembrane domain.

10. The immune cell of any one of claims 1-9, wherein a hinge region of the CAR comprises a CD28 hinge region.

11. The immune cell of any one of claims 1-10, wherein an intracellular signaling domain of the CAR comprises a CD28 intracellular signaling domain or a CD3-zeta intracellular signaling domain.

12. The immune cell of any one of claims 1-11, wherein a transmembrane domain of the iCAR comprises a CD8-alpha transmembrane domain, a PD-1 transmembrane domain, or a CTLA-4 transmembrane domain.

13. The immune cell of any one of claims 1-12, wherein the hinge region of the iCAR comprises a CD8-alpha hinge region, a PD-1 hinge region, or a CTLA-4 hinge region.

14. The immune cell of any one of claims 1-13, wherein the intracellular signaling domain of the iCAR comprises a PD-1 inhibitory domain or a CTLA-4 inhibitory domain.

15. The immune cell of any one of claims 1-14, wherein the CAR and the iCAR are conditionally expressed in the immune cell.

16. The immune cell of claim 15, wherein the CAR and the iCAR are expressed in the immune cell in a tumor microenvironment.

17. The immune cell of claim 16, wherein the immune cell further comprises a synNotch receptor, wherein the synNotch receptor activates the CAR and the iCAR expression when the immune cell is in the tumor microenvironment.

18. A pharmaceutical composition comprising an immune cell of any one of claims 1-17 and a pharmaceutically acceptable carrier.

19. A method of treating a subject having a disease, the method comprising administering to the subject an immune cell of any one of claims 1-17, or a pharmaceutical composition of claim 18.

20. The method of claim 19, wherein the disease is a cancer.

21. The method of claim 19, wherein the disease is a precancerous pathology with LOH.

22. The method of claim 20, wherein the subject has previously been administered one or more additional anticancer therapies selected from the group consisting of ionizing radiation, a chemotherapeutic agent, a therapeutic antibody, or a checkpoint inhibitor.

23. The method of claim 20, wherein the subject will be administered one or more additional anticancer therapies selected from the group consisting of ionizing radiation, a chemotherapeutic agent, a therapeutic antibody, or a checkpoint inhibitor.

24. The method of claim 20, wherein the cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.

25. The method of claim 21, wherein the precancerous pathology is selected from a myelodysplastic syndrome, acquired aplastic anemia, Fanconi anemia, paroxysmal nocturnal hemoglobinuria (PNH), 5q-syndrome, or any condition characterized by pathogenic cells with clonal LOH.

26. The method of claim 19, wherein the immune cell targets LOH present in a hematologic malignancy.

27. The method of claim 26, wherein the hematologic malignancy is selected from myelodysplastic syndrome (MDS), acute myeloid leukemia (AMIL), or acute lymphocytic leukemia (ALL).

28. The method of claim 26, wherein the hematologic malignancy has relapsed after matched, mis-matched and haploidentical blood or marrow transplantation.

29. The method of claim 19, wherein the immune cell targets LOH which are present in cancer cells and cause relapse after targeted CAR T cell therapy or bispecific antibody therapy directed against a tumor associated antigen.

30. The method of claim 29, wherein the tumor associated antigen is CD19 or NY-ESO-1.
